(19) United States
(12) Patent Application Publication
(10) Pub. No.: US 2013/0133679 A1
(43) Pub. Date: May 30, 2013

(54) COSMETIC TREATMENT PROCESS USING A COATING BASED ON A COPOLYMER CONTAINING POLYAMIDE BLOCKS AND POLYETHER BLOCKS

(71) Applicants: Guillaume Kergosien, Chaville (FR); Henri Samain, Bievres (FR)

(72) Inventors: Guillaume Kergosien, Chaville (FR); Henri Samain, Bievres (FR)

(21) Appl. No.: 13/701,121

(22) PCT Filed: May 20, 2011
(86) PCT No.: PCT/EP11/58268

§ 371 Date: Feb. 7, 2013

Related U.S. Application Data
(60) Provisional application No. 61/353,543, filed on Jun. 10, 2010.

(30) Foreign Application Priority Data
Jun. 3, 2010 (FR) 1054337

(51) Int. Cl. A61K 8/90; A61Q 19/00

(52) U.S. Cl. CPC A61K 8/90
USPC 132/200; 514/772.1; 424/70.17; 8/405; 8/406; 424/63; 424/64; 424/61; 424/70.7; 424/70.6; 424/59; 424/65

(57) ABSTRACT

The present invention relates to a cosmetic process for treating a keratin material, characterized in that it comprises at least one step of applying to the said keratin material a coating formed from a liquid composition comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent. The present invention also relates to a cosmetic assembly comprising a) at least one liquid composition comprising, in a cosmetically acceptable medium, at least one copolymer containing polyamide blocks and polyether blocks in solution or in dispersion, and at least one organic solvent; b) a device for applying the said composition to the keratin materials.

The present invention relates to a process for the cosmetic treatment of a keratin material involving at least the application to the said keratin material of a coating formed in situ from a liquid composition comprising, in a cosmetically acceptable medium, at least one copolymer containing polyamide blocks and polyether blocks in solution or in dispersion, and at least one organic solvent.

The present invention also relates to a cosmetic assembly comprising
a) at least one liquid composition comprising, in a cosmetically acceptable medium, at least one copolymer containing polyamide blocks and polyether blocks in solution or in dispersion, and at least one organic solvent;
b) a device for applying the said composition to the keratin materials.

The object of the present invention is to produce novel cosmetic coatings for keratin materials, especially for the skin, the lips, the hair, the eyelashes, the eyebrows and the nails, which are resistant to these substrates, which can be spread easily and uniformly over the entire area to be treated, which do not produce any tautness, which are insensitive, which do not produce a gloss effect, which are resistant to movements and to friction and which are water-resistant, without limiting exchanges of gases such as water vapour or oxygen so as to allow the keratin materials thus coated to breathe.

Another object of the invention is also to find coatings that are easy to remove.

Another object of the invention is also to find materials that are capable of forming a coating with which several levels of thickness can be produced.

Another object of the invention is also to find coatings that are of low thickness without losing their resistance properties: in particular less than 10 μm.

In order to allow good spreading of the materials capable of giving a coating, formulations comprising the said material in solution or in dispersion with one or more solvents are generally used. The fastest possible drying is sought so as not to oblige the person to touch the coated keratin areas such as the skin during long drying. The cosmetic coatings produced are generally sparingly resistant and are unsuitable for use.

If the material capable of forming the coating is too rigid, the coating thus obtained has a tendency to pull on the covered keratin material, in particular the skin. To solve the problems of tautness, very thin coatings are produced, using small concentrations of material. However, the coating thus obtained has a tendency to not withstand movements or friction, or even is not uniform (there are holes). Holes are harmful to the remanence of the coating since they form points of detachment.

To overcome the problems of tautness, a material capable of forming a coating that is elastomeric may also be used. However, usually, the coating thus formed adheres poorly to the keratin material and is not resistant for long. In particular, if the coating is thin, it detaches even more quickly, or even becomes fragmented. Furthermore, elastomeric materials in solution are often tacky. This gives discomfort effects that are difficult to overcome. It is also possible to select elastomeric materials in dispersion (latex), but, to obtain a film, the coalescence rules oblige the use of materials that will pose problems once the coating has been produced: somewhat tacky feel, or gloss.

To overcome these drawbacks, formulators of cosmetic coating materials have turned towards coatings based on fatty substances or towards techniques for forming a coating via an in situ chemical reaction.

Materials of the first type based on fatty substances, which are widely used, have the advantage of being easy to spread. However, this type of coating shows limited resistance, especially to friction. They give good results for producing foundations, but are unsuitable for producing very resistant coatings, in particular waterproof coatings.

The second types give advantageous coatings, but are limited to the treatment of small areas. Furthermore, they give coatings that are poorly adherent, and as a result are sparingly in demand.

In the course of its investigations, the Applicant has discovered, surprisingly, that the objectives stated previously can be achieved by using a coating formed from a liquid composition comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent.

The present invention relates to a cosmetic process for treating a keratin material, characterized in that it comprises at least one step of applying to the said keratin material a coating formed from a liquid composition comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent.

The present invention also relates to a cosmetic assembly comprising
c) at least one liquid composition comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent;
d) a device for applying the said composition to the keratin material to be treated.

The term “cosmetically acceptable” means compatible with the skin, the lips, the nail, the scalp, the hair, the eyelashes, the eyebrows and mucous membranes, which has a pleasant colour, odour and feel and which does not give rise to unacceptable discomfort liable to discourage the consumer from using this composition.

The term “keratin material” means the skin, the lips, the nails, the scalp or the integuments: hair, eyelashes, eyebrows and mucous membranes.

The term “liquid composition” means a composition that is not in a solid form at room temperature (25° C.) and atmospheric pressure (760 mmHg). The compositions in accordance with the invention comprising the polyamide-polyether polymer are preferably in the form of an organic solution, an aqueous-organic solution, an organic dispersion, an aqueous-organic dispersion or an emulsion comprising an aqueous phase and a water-immiscible organic phase (macroemulsion, microemulsion or nanoemulsion).

The term “polymer in solution” means that the polymer is soluble in the organic solvent or a mixture of water and of the said organic solvent used in the liquid composition of the invention to a solids content of 1% by weight in the said solvent, at room temperature (25° C., 1 atmosphere), so as to obtain a transparent solution, namely a solution with a minimum light transmittance at 500 nm, through a sample 1 cm thick, of greater than 80%, or even 90%.

The term “polymer in dispersion” means that the polymer is dispersible in the organic solvent or a mixture of water and of the said organic solvent used in the liquid composition of the invention to a solids content of 1% by weight in the said solvent, at room temperature (25° C., 1 atmosphere), so as to obtain a dispersion of polymer particles with a mean size from 5 nm to 5 μm.

The term “copolymer containing polyamide blocks and polyether blocks” means any block copolymer comprising at least two polyamide blocks and at least two polyether blocks.

### Polyamide-Polyether Polymers

The copolymers containing polyamide blocks and polyether blocks used in accordance with the present invention are preferably capable of leading, by drying at room temperature and at a relative humidity of 55%, to a material with a mechanical profile defined by at least:
a degree of elongation at break (ε) of greater than or equal to 150%,
an instantaneous recovery (Rᵢ) of greater than or equal to 75% after an elongation of 150%,
a recovery at 300 seconds (R₃₀₀ₛ) of greater than 80%, after an elongation of 150%.

For the purposes of the present invention, the term “relative humidity” means the ratio of the partial vapour pressure of water vapour contained in the air to the saturating vapour pressure at the same temperature and pressure. This value makes it possible to measure the ratio between the water vapour content of the air and its maximum capacity for containing water under these conditions. The relative humidity is measured using a hygrometer.

For the purposes of the present invention, the elongation at break (c) of a material defines its capacity to be stretched before breaking when it is placed under a tensile stress. The degree of elongation of the material is measured as a percentage.

For the purposes of the present invention, the instantaneous recovery (Ri) of a material defines its capacity to regain its initial shape or a shape substantially identical to its initial shape after having been deformed following an elongation during a tensile stress. The recovery of the material is also measured as a percentage.

For the purposes of the present invention, the degree of elongation at break and the recovery are evaluated by means of the tensile tests described below.

To perform the tensile tests, a film intended for producing specimens is made by placing in a Teflon mould a sufficient amount of mixture comprising the film-forming elastomeric polymer(s) to obtain a film 500 μm μ150 μM thick. Drying is continued until the weight of the film no longer changes, which may typically take 12 days.

In particular, for the purposes of the present invention, the term “film intended for producing or making specimens” means a film obtained by drying the said film-forming elastomeric polymer(s), at room temperature (22° C.±2° C.) and at a relative humidity of 55%±5%, from a mixture containing at least 3% of active materials, i.e. 3% by weight of film-forming elastomeric polymer relative to the total weight of the mixture.

When the mixture used to produce the film for the manufacture of specimens contains less than 3% by weight of active materials, a preliminary concentration operation is performed, for example by evaporating off some of the solvent so that the mixture contains at least 3% of elastomeric polymers. This operation makes it possible to avoid excessively long drying.

The film obtained is then chopped into rectangular specimens 80 mm long and 15 mm wide.

The tests are performed on a machine sold under the name Lloyd or sold under the name Zwick, under the same temperature and humidity conditions as for the drying, i.e. at room temperature (22° C.±2° C.) and at a relative humidity of 55%±5%.

The specimens are drawn at a rate of 20 mm/minute and the distance between the jaws is 50±1 mm.

To determine the instantaneous recovery (Rᵢ), the process is performed as follows:
the specimen is drawn by 150% (εmax), i.e. 1.5 times its initial length (10),
the stress is removed by imposing a return speed equal to the tensile speed, i.e. 20 mm/minute, and the elongation of the specimen is measured as a percentage, after returning to zero load (εi).

The percentage instantaneous recovery (Ri) is given by the formula below:

Rᵢ=((εₘₐₓ−εᵢ)/εₘₐₓ)×100

To determine the recovery at 300 seconds, the specimen is maintained at zero stress for a further 300 seconds, after having undergone the preceding operations, and its degree of elongation is measured as a percentage (ε300 s). In other words, the recovery at 300 seconds corresponds to the residual degree of elongation of the specimen 300 seconds after returning to zero load (εi).

Thus, the recovery at 300 seconds (R₃₀₀ₛ) of a material defines its capacity to regain its shape or a shape substantially identical to its initial shape a further 300 seconds after the return to zero load (εi) and after having been deformed following an elongation during a tensile stress.

The percentage recovery at 300 seconds (R₃₀₀ₛ) is therefore given by the formula below:

R₃₀₀ₛ=((εₘₐₓ−εᵢ)/ε₃₀₀ₛ)×100

Preferably, the polymers of polyamide-polyether type have a tensile strength characterized by a breaking force (ASTM D 638) of greater than 20 MPa, preferably greater than 25 MPa and preferably greater than 30 MPa.

Preferably also, the copolymer containing polyamide blocks and polyether blocks of the invention will be water-insoluble.

According to one particular mode of the invention, the copolymers containing polyamide blocks and polyether blocks of the invention have a permeability to water vapour of greater than 1000 g/m²/24 hours. This permeability is determined by measuring the amount of water vapour in grams that crosses 1 m² of coating in 24 hours, from a wet zone to a dry zone (ASTM E 96 E). The operating conditions adopted are a relative humidity percentage of 90% and a temperature of 38° C. The measurement is performed on a sample of coating 15 μm thick. The permeability measurement may be measured using permeability-measuring equipment such as the equipment bearing the reference Perme™ W3-060 available from the company Labthink Instruments Co. Limited.

According to another particular mode of the invention, the copolymer containing polyamide blocks and polyether blocks of the invention may be characterized by adhesion properties on the skin higher than the adhesion properties obtained with conventional water-resistant elastomeric polymers such as certain dispersions of polyurethane in aqueous phase available especially under the reference Baycusan C1001 from Bayer.

The copolymers containing polyamide blocks and polyether blocks of the invention will preferably be chosen from copolymers containing polyamide-6 or polyamide-12 blocks and containing polyether blocks of the polyethylene glycol or polytetramethylene glycol type.

The copolymers containing polyamide blocks and polyether blocks according to the invention result from the copolycondensation of polyamide blocks bearing reactive ends with polyether blocks bearing reactive ends, such as, inter alia:

1) Polyamide blocks bearing diamine chain ends with polyoxyalkylene blocks bearing dicarboxylic chain ends.
2) Polyamide blocks bearing dicarboxylic chain ends with polyoxyalkylene blocks bearing diamine chain ends obtained by cyanoethylation and hydrogenation of α,ω-dihydroxylated aliphatic polyoxyalkylene blocks known as polyether diols.
3) Polyamide blocks bearing dicarboxylic chain ends with polyether diols, the products obtained being, in this particular case, polyetheresteramides. The copolymers of the invention are advantageously of this type. The polyamide blocks bearing dicarboxylic chain ends are derived, for example, from the condensation of polyamide precursors in the presence of a chain-limiting dicarboxylic acid.

The polyamide blocks bearing diamine chain ends originate, for example from the condensation of polyamide precursors in the presence of a chain-limiting diamine.

The polymers bearing polyamide blocks and polyether blocks of the invention may also comprise randomly distributed units. These polymers may be prepared by the simultaneous reaction of the polyether and of the precursors of the polyamide blocks.

For example, it is possible to react polyether diol, polyamide precursors and a chain-limiting diacid. A polymer is obtained essentially having polyether blocks and polyamide blocks of very variable length, but also the various reagents that have reacted randomly, which are distributed randomly (statistically) along the polymer chain.

It is also possible to react polyetherdiamine, polyamide precursors and a chain-limiting diacid. A polymer is obtained essentially having polyether blocks and polyamide blocks of very variable length, but also the various reagents that have reacted randomly, which are distributed randomly (statistically) along the polymer chain.

Three types of polyamide block may advantageously be used.

According to a first type, the polyamide blocks originate from the condensation of a dicarboxylic acid and a diamine.

According to a second type, the polyamide blocks result from the condensation of one or more α,ω-aminocarboxylic acids and/or of one or more lactams containing from 6 to 12 carbon atoms in the presence of a dicarboxylic acid containing from 4 to 12 carbon atoms or of a diamine.

According to a third type, the polyamide blocks result from the condensation of at least one α,ω-aminocarboxylic acid (or a lactam), at least one diamine and at least one dicarboxylic acid.

According to one variant of this third type, the polyamide blocks result from the condensation of at least two α,ω-aminocarboxylic acids or from at least two lactams containing from 6 to 12 carbon atoms or from one lactam and one aminocarboxylic acid not having the same number of carbon atoms, in the optional presence of a chain limiter. Advantageously, the polyamide blocks of the second type are made of polyamide-12 or polyamide-6. As examples of polyamide blocks of the third type, mention may be made of the following:

a) 6.6/Pip. 10/12 in which 6.6 denotes hexamethyleneadipamide units (hexamethylenediamine condensed with adipic acid). Pip. 10 denotes units resulting from the condensation of piperazine and of sebacic acid. 12 denotes units resulting from the condensation of lauryllactam. The weight proportions are, respectively, 25 to 35/20 to 30/20 to 30; the total being 80, and advantageously 30 to 35/22 to 27/22 to 27; the total being 80. For example, 32/24/24 proportions give a melting point from 122 to 137° C.
b) 6.6/6.10/11/12 in which 6.6 denotes hexamethylenediamine condensed with adipic acid, 6.10 denotes hexamethylenediamine condensed with sebacic acid, and 11 denotes units resulting from the condensation of aminoundecanoic acid, and 12 denotes units resulting from the condensation of lauryllactam. The weight proportions are, respectively, 10 to 20/15 to 25/10 to 20/15 to 25; the total advantageously being 70:12 to 16/18 to 25/12 to 16/18 to 25; the total being 70. For example, 14/21/14/21 proportions give a melting point from 119 to 131° C.

The polyamide blocks are obtained in the presence of a diacid or of a chain-limiting diamine if polyamide blocks bearing acid or amine ends are desired. If the precursors already comprise a diacid or a diamine, it suffices, for example, to use it in excess. By way of example of aliphatic α,ω-aminocarboxylic acids, mention may be made of aminocaproic acid, 7-aminoheptanoic acid, 11-aminoundecanoic acid and 12-aminododecanoic acid. As examples of lactams, mention may be made of caprolactam, oenantholactam and lauryllactam. As examples of aliphatic diamines, mention may be made of hexamethylenediamine, dodecamethylenediamine and trimethylhexamethylenediamine. As an example of a cycloaliphatic diacid, mention may be made of 1,4-cyclohexyldicarboxylic acid. As examples of aliphatic diacids, mention may be made of butanedioic acid, adipic acid, azelaic acid, suberic acid, sebacic acid, dodecanedicarboxylic acid, dimerized fatty acids (these dimerized fatty acids preferably have a dimer content of at least 98%; they are preferably hydrogenated; they are sold under the brand name Pripol by the company Unichema, or under the brand name Empol by the company Henkel) and polyoxyalkylene-am-diacids. As examples of aromatic diacids, mention may be made of terephthalic acid (T) and isophthalic acid (I). The cycloaliphatic diamines may be bis(4-aminocyclohexyl)methane (BACM), bis(3-methyl-4-aminocyclohexyl)methane (BMACM), 2-2-bis(3-methyl-4-aminocyclohexyl)propane (BMACP) and para-aminodicyclohexylmethane (PACM) isomers. The other diamines commonly used may be isophoronediamine (IPDA), 2,6-bis(aminomethyl)norbornane (BAMN) and piperazine.

The polyether blocks may represent 5% to 85% by weight of the copolymer bearing polyamide and polyether blocks.

The polyether blocks are formed from alkylene oxide units. These units may be, for example, ethylene oxide units, propylene oxide units or tetrahydrofuran (which leads to polytetramethylene glycol chains). Use is thus made of PEG blocks, i.e. blocks formed from ethylene oxide units, PPG blocks, i.e. blocks formed from propylene oxide units, polytrimethylene glycol ether units (such copolymers with polytrimethylene ether blocks are described in U.S. Pat. No. 6,590,065), and PTMG blocks, i.e. blocks formed from tetramethylene glycol units, also known as polytetrahydrofuran.

Use is advantageously made of PEG blocks or of blocks obtained by oxyethylation of bisphenols, for instance bisphenol A. The latter products are described in patent EP 613 919.

The polyether blocks may also be formed from ethoxylated primary amines.

These blocks are also advantageously used. As examples of ethoxylated primary amines, mention may be made of the products of formula:

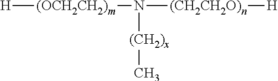

in which m and n are between 1 and 20, and x is between 8 and 18. These products are available commercially under the brand name Noramox® from the company Ceca and under the brand name Genamin® from the company Clariant.

The amount of polyether blocks in these copolymers bearing polyamide blocks and polyether blocks is advantageously from 10% to 70% by weight and preferably from 35% to 60% by weight of the copolymer.

The polyether diol blocks are either used in unmodified form and copolycondensed with polyamide blocks bearing carboxylic end groups, or they are aminated to be converted into polyetherdiamines and condensed with polyamide blocks bearing carboxylic end groups. They may also be mixed with polyamide precursors and a chain-limiting diacid to make polymers bearing polyamide blocks and polyether blocks having randomly distributed units.

The number-average molar mass Mn of the polyamide sequences is between 500 and 10 000 and preferably between 500 and 4000, except for the polyamide blocks of the second type.

The mass Mn of the polyether blocks is between 100 and 6000 and preferably between 200 and 3000.

These polymers bearing polyamide blocks and polyether blocks, whether they originate from the copolycondensation of polyamide and polyether blocks prepared previously or from a one-step reaction, have, for example, an intrinsic viscosity of between 0.8 and 2.5 measured in meta-cresol at 25° C. for an initial concentration of 0.8 g/100 ml.

As regards the preparation of the copolymers bearing polyamide blocks and polyether blocks, they may be prepared via any means for attaching polyamide blocks and polyether blocks.

In practice, essentially two processes are used, one known as a two-step process, the other a one-step process.

In the two-step process, the polyamide blocks are first made, and in a second step the polyamide blocks and the polyether blocks are then attached.

In the one-step process, the polyamide precursors, the chain limiter and the polyether are mixed together; a polymer essentially having polyether blocks and polyamide blocks of very variable length, but also the various reagents that have reacted randomly, which are distributed randomly (statistically) along the polymer chain is obtained.

Whether it is a one-step or a two-step process, it is advantageous to perform the process in the presence of a catalyst. The catalysts described in U.S. Pat. No. 4,331,786, U.S. Pat. No. 4,115,475, U.S. Pat. No. 4,195,015, U.S. Pat. No. 4,839,441, U.S. Pat. No. 4,864,014, U.S. Pat. No. 4,230,838 and U.S. Pat. No. 4,332,920, WO 04/037 898, EP 1 262 527, EP 1 270 211, EP 1 136 512, EP 1 046 675, EP 1 057 870, EP 1 155 065, EP 506 495 and EP 504 058 may be used. Polyamide blocks are also manufactured in the one-step process.

Copolymers bearing polyamide blocks (in particular PA-6 or PA-12) and polyether blocks (in particular polyethylene glycol or polytetramethylene glycol ether) and more particularly those sold under the name Pebax® by the company Arkema, will be more particularly used. Preferably, the polymer is chosen from the references Pebax® 2533 SA 01, Pebax® 2533 SD 02, Pebax® 3533 SA 01, and Pebax® 2533 SP 01. Preferably, the polymer according to the invention is the reference Pebax® 2533 SA 01.

In the compositions in accordance with the invention, the copolymer(s) containing polyamide blocks and polyether blocks are preferably present in a concentration ranging from 0.05% to 20% by weight, more preferentially from 0.1% to 15% by weight and, for example, from 0.25% to 10% by weight relative to the total weight of the composition. The amounts will vary as a function of the desired cosmetic application.

### Organic Solvents

The organic solvent(s) used in the presence of the copolymer(s) containing polyamide blocks and polyether blocks may be chosen from linear C₁-C₄ monoalcohols, in particular ethanol; C₁-C₃₀ alkanes such as propane, butane, isobutane or mixtures thereof, isododecanes, paraffins; acetone, liquefied gases such as dimethyl ether.

According to one particular form of the invention, the organic solvent(s) may also be one or more propellants in an aerosol device, which, at the time of use and from the formulation outlet, dissolve the polyamide-polyether polymer as defined previously.

Mention may also be made of C₈-C₁₄ fatty alcohols that are liquid at room temperature, solvents of the fatty amide type, such as isopropyl N-lauroylsarcosinate (Eldew SL-205 by the company Ajinomoto), and C₃-C₈ alkylene carbonates, such as propylene carbonate.

In the compositions in accordance with the invention, the organic solvent(s) are preferably present in a concentration ranging from 40% to 99% by weight, more preferentially from 60% to 97% by weight, for example from 80% to 95% by weight, relative to the total weight of the composition. The amounts will vary as a function of the desired cosmetic application.

Agents that Aid or Modify the Coating Properties

The compositions of the invention comprising the copolymer(s) containing polyamide blocks and polyether blocks may also comprise at least one agent that aids or modifies the coating properties, chosen especially from plasticizers, film-forming agents, coalescers, film-forming polymers other than those of the invention, fillers, fibres, crosslinking agents, crosslinkable polymers or reactive monomers such as the cyanoacrylates mentioned in patent application EP 1 649 894.

As examples of plasticizers and/or agents for facilitating the formation of a film on the surface of a human keratin material, those described in document FR-A-2 782 917 may be used.

It is especially possible to use standard plasticizers or coalescers such as:
glycols and derivatives thereof such as diethylene glycol ethyl ether, diethylene glycol methyl ether, diethylene glycol butyl ether or diethylene glycol hexyl ether, ethylene glycol ethyl ether, ethylene glycol butyl ether, ethylene glycol hexyl ether or pentylene glycol, glycerol esters,
propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
acid esters, especially carboxylic acid esters, such as citrates, phthalates, adipates, carbonates, tartrates, phosphates, sebacates, or mineral coalescers such as zinc, aluminium, calcium, magnesium or manganese salts,
mixtures thereof.

The agent(s) for aiding or modifying the coating properties may also give the coating obtained a water-impermeable nature.

The amount of agent for aiding or modifying the coating properties may be chosen by a person skilled in the art on the basis of his general knowledge, so as to obtain a polymeric system leading to a film having the desired mechanical properties, while at the same time conserving the composition's desired cosmetic properties. In practice, this amount ranges from 0.01% to 25% of the total weight of the composition and better still from 0.01% to 15% relative to the total weight of the composition.

### Coating

The liquid compositions in accordance with the invention comprising the copolymer(s) containing polyamide blocks and polyether blocks will preferably form in situ, after drying, a film on the surface of the keratin material to be treated.

The term “film” means a thin, manipulable solid. The term “thin” means a solid with a thickness of at least 1 μM and even more preferentially of at least 5 μm. Such a film may have a square, rectangular or disc shape, or any other shape.

The film thus obtained under these conditions may have, depending on the intended use, a thickness from 1 μm to 1000 μm, preferably from 4 to 200 μm and better still from 8 to 100 μm. It may have an area from 10 to 800 cm² and preferably from 40 cm² to 200 cm².

In certain applications, especially for the skin, it is sought to prepare films that have a thickness ranging from 1 μm to 10 μm and even more preferentially from 1 to 6 μm. Such a film may have a square, rectangular or disc shape, or any other shape.

### Cosmetic Applications

According to one particular mode of the invention, the cosmetic treatment process in accordance with the invention may include an additional step for bringing about the drying of the copolymer(s) containing polyamide blocks and polyether blocks, for instance the application of a supply of energy such as a hairdryer or an iron (for the hair), the application of a liquid vapour mixture (especially for the hair), the application of infrared, of ultrasonication, of microwaves, of vibrations or of rotational movements.

According to another particular embodiment of the invention, the cosmetic treatment process in accordance with the invention may include an additional step for facilitating the removal of the coating, for instance the application of solubilizers, surfactants, powders or compounds that are reactive with the polymer(s) constituting the coating.

According to another particular embodiment of the invention, the cosmetic treatment process in accordance with the invention may include

(i) a step of pretreatment of the keratin materials and/or
(ii) a step of pretreatment of the composition containing the copolymer containing polyamide blocks and polyether blocks, for instance heating, for example in order to fluidize the composition, stirring, for instance centrifugation, and redispersion.

According to one particular embodiment of the invention, the cosmetic process for treating a keratin material may consist in

a) applying to the said human keratin material a cosmetic composition (A) comprising at least one agent for treating keratin materials;
b) optionally rinsing and/or drying;
c) applying to the said keratin material coating formed in situ from a liquid composition (B) comprising, in a cosmetically acceptable medium, at least the copolymer containing polyamide blocks and polyether blocks in solution or in dispersion, and at least one organic solvent.

By way of example, to illustrate this particular cosmetic treatment process, the skin, the lips, the nails, the hair, the eyelashes or the eyebrows may be dyed with a first dye composition (A) and the colorant(s) applied to the skin may be taken up by applying the coating formed in situ on the skin from composition (B).

In one particular embodiment of the invention, using a water-impermeable coating that is permeable to water vapour, a water-sensitive active compound may be introduced into composition (A). This compound is applied, for example, to the skin in the first step. Next, the coating with composition (B) is applied. Thereafter, the active compound may be dissolved or activated by the action of perspiration water, atmospheric water or added water.

According to another particular form of the invention, the cosmetic process for treating a human keratin material may consist in

a) applying to the said keratin material a coating formed in situ from a liquid composition (B) comprising, in a cosmetically acceptable medium, at least the copolymer containing polyamide blocks and polyether blocks in solution or in dispersion, and at least one organic solvent;
b) optionally rinsing and/or drying;
c) applying to the said keratin material a cosmetic composition (A) comprising at least one agent for treating keratin materials.

By way of example, to illustrate this particular cosmetic treatment process, the skin or the hair may be protected by applying the coating formed in situ on the skin from composition (B) before colouring the skin or the hair, to avoid staining outside the area to be coloured.

Another subject of the invention is thus a cosmetic assembly comprising at least:
(i) a first composition (A) as defined previously;
(ii) a second composition (B) as defined previously.

In one particular embodiment of the invention, using a water-impermeable coating that is permeable to water vapour, a water-sensitive active compound may be introduced into composition (B). The water-sensitive active agent may react, become dissolved or become hydrated without any risk of being removed since it is taken up by the coating. This process is particularly suited to reactive compounds such as reducing agents, oxidizing agents, crosslinkable compounds, salts, dyes and fluorescers.

The agents for treating keratin materials that may be used according to the various cosmetic treatment processes of the invention are diverse. They may be chosen especially from haircare agents, such as antidandruff agents, hair conditioners, agents that participate in dyeing their hair, such as oxidation dye precursors, direct dyes, reducing agents and oxidizing agents; skincare active agents, especially sunscreens, antiperspirants, deodorants, agents for colouring the skin, the lips, the face, the nails, the eyelashes or the eyebrows, for instance self-tanning agents, pigments, nacres or direct dyes; sheen agents or matting agents, agents for producing optical effects, fluorescers; fragrances.

The present invention also relates to a cosmetic assembly comprising
a) at least one liquid composition as defined previously, comprising, in a cosmetically acceptable medium, at least one copolymer containing polyamide blocks and polyether blocks in solution or in dispersion, and at least one organic solvent;
b) a device for applying the said composition to the keratin material to be treated.

The application devices that may be used according to the invention are chosen from devices that are suitable for hair use or for use in making up or caring for the skin, the body, the lips, the eyelashes, the eyebrows or the nails. Mention may be made especially of devices comprising the liquid composition in pressurized form, such as aerosols, sprays and various types of spraying devices. Mention may also be made of brushes, spatulas, applicators in foam formed, which may be flocked, sponges, wipes, brushes, pads, roll-ons, etc.

I/Hair Uses

### 1) Hairstyling

The present invention also relates to a process for shaping and/or holding the hair, which consists in applying to the hair a coating formed in situ from a liquid composition as defined previously, comprising, in a cosmetically acceptable medium, at least one copolymer containing polyamide blocks and polyether blocks in solution or in dispersion, and at least one organic solvent.

### 2) Antidandruff Treatment

The present invention also relates to an antidandruff cosmetic treatment process, characterized in that a coating formed in situ from a liquid composition (B) comprising, in a cosmetically acceptable medium, at least one copolymer containing polyamide blocks and polyether blocks in solution or in dispersion, at least one organic solvent and at least one antidandruff agent is applied to the scalp and/or the hair.

A subject of the present invention is also an antidandruff cosmetic treatment process, characterized in that

1) a composition (A) comprising, in a cosmetically acceptable medium, at least one antidandruff agent is applied to the scalp and/or the hair
2) optionally, rinsing and/or washing with shampoo and/or partial or total drying of the scalp and/or of the hair is then performed,
3) a coating formed in situ from a liquid composition (B) comprising, in a cosmetically acceptable medium, at least the copolymer containing polyamide blocks and polyether blocks in solution or in dispersion, and at least one organic solvent is applied to the said human keratin material.

Another subject of the invention is thus a cosmetic assembly comprising at least:
(i) a first composition (A) as defined previously;
(ii) a second composition (B) as defined previously.

The antidandruff agents may be chosen especially from zinc pyrithione, selenium sulfide, piroctone olamine (Octopyrox from Clariant International), or mixtures thereof.

### 2) Hair Dyeing

A subject of the present invention is also a hair dyeing process, comprising the following steps

1) a direct or oxidation dye composition (A) is applied to the said hair for a time sufficient to develop the colour,
2) optionally, rinsing and/or washing with shampoo and/or partial or total drying of the hair is then performed,
3) a coating formed in situ from a liquid composition (B) comprising, in a cosmetically acceptable medium, at least the copolymer containing polyamide blocks and polyether blocks in solution or in dispersion, and at least one organic solvent is applied to the said human keratin material.

A subject of the present invention is also a hair dyeing process, comprising the following steps

1) a coating formed in situ from a liquid composition (B) comprising, in a cosmetically acceptable medium, at least the copolymer containing polyamide blocks and polyether blocks in solution or in dispersion, and at least one organic solvent is applied to the said human keratin material,
2) optionally, rinsing and/or washing with shampoo and/or partial or total drying of the hair is then performed,
3) a direct or oxidation dye composition (A) is applied to the said hair for a time sufficient to develop the colour.

According to a first variant, a liquid water/water vapour mixture whose temperature is at least 35° C. may also be applied to the hair. This step may be performed after step 1 or after step 2.

According to a second variant, a heating iron whose temperature is greater than or equal to 60° C. may also be applied to the hair. This step is performed after the application of the dye composition (A) or of the coating formed from composition (B).

The nature and concentration of the dyes present in the dye composition (A) is not critical.

In the case of lightening direct dyeing, the dye compositions (A) result from the mixing at the time of use of a dye composition (A₁) containing one or more direct dyes and of a composition (A₂) containing one or more oxidation dyes.

In the case of oxidation dyeing, the dye compositions (A) result from the mixing at the time of use of a dye composition (A₁) containing one or more oxidation bases and optionally one or more couplers and/or one or more direct dyes and of a composition (A₂) containing one or more oxidation dyes. In one particular case of the invention, the compounds may be self-oxidizable, i.e. becoming oxidized on contact with air. In this case, compounds such as aromatic compounds bearing one or more hydroxyl functions, such as catechol, 5,6-dihydroxyindole or natural polyphenols are used.

A subject of the invention is also a multi-component dyeing agent or kit comprising a first component comprising a direct dye composition (A) and a second component comprising a composition (B) as defined previously.

A subject of the invention is also a multi-component dying agent or kit comprising a first component comprising a composition (A₁) comprising one or more direct dyes, a second component comprising a composition (A₂) containing one or more oxidizing agents and a third component comprising a composition (B) as defined previously.

A subject of the invention is also a multi-component dying agent or kit comprising a first component comprising a composition (A₁) comprising one or more oxidation dye precursors, a second component comprising a composition (A₂) containing one or more oxidizing agents and a third component comprising a composition (B) as defined previously.

### Direct Dyes

As regards the direct dyes, these dyes are more particularly chosen from ionic and nonionic species, preferably cationic or nonionic species.

Examples of suitable direct dyes that may be mentioned include azo dyes; methine dyes; carbonyl dyes; azine dyes; nitro (hetero)aryl dyes; tri(hetero)arylmethane dyes; porphyrin dyes; phthalocyanine dyes, and natural direct dyes, alone or as mixtures.

More particularly, the azo dyes comprise an —N═N— function in which the two nitrogen atoms are not simultaneously engaged in a ring. However, it is not excluded for one of the two nitrogen atoms of the sequence —N═N— to be engaged in a ring.

The dyes of the methine family are more particularly compounds comprising at least one sequence selected from >C═C< and —N═C< in which the two atoms are not simultaneously engaged in a ring. However, it is pointed out that one of the nitrogen or carbon atoms of the sequences may be engaged in a ring. More particularly, the dyes of this family are derived from compounds of the type such as methines, azomethines, mono- and diarylmethanes, indoamines (or diphenylamines), indophenols, indoanilines, carbocyanins, azacarbocyanins and isomers thereof, diazacarbocyanins and isomers thereof, tetraazacarbocyanins and hemicyanins.

As regards the dyes of the carbonyl family, examples that may be mentioned include dyes chosen from acridone, benzoquinone, anthraquinone, naphthoquinone, benzanthrone, anthranthrone, pyranthrone, pyrazolanthrone, pyrimidinoanthrone, flavanthrone, idanthrone, flavone, (iso)violanthrone, isoindolinone, benzimidazolone, isoquinolinone, anthrapyridone, pyrazoloquinazolone, perinone, quinacridone, quinophthalone, indigoid, thioindigo, naphthalimide, anthrapyrimidine, diketopyrrolopyrrole and coumarin.

As regards the dyes of the cyclic azine family, mention may be made especially of azine, xanthene, thioxanthene, fluorindine, acridine, (di)oxazine, (di)thiazine and pyronin.

The nitro (hetero)aromatic dyes are more particularly nitrobenzene or nitropyridine direct dyes.

As regards the dyes of porphyrin or phthalocyanin type, it is possible to use cationic or non-cationic compounds, optionally comprising one or more metals or metal ions, for instance alkali metals, alkaline-earth metals, zinc and silicon.

Examples of particularly suitable direct dyes that may be mentioned include nitrobenzene dyes; azo direct dyes; azomethine direct dyes; methine direct dyes; azacarbocyanin direct dyes, for instance tetraazacarbocyan ins (tetraazapentamethines); quinone and in particular anthraquinone, naphthoquinone or benzoquinone direct dyes; azine direct dyes; xanthene direct dyes; triarylmethane direct dyes; indoamine direct dyes; indigoid direct dyes; phthalocyanine direct dyes, porphyrin direct dyes and natural direct dyes, alone or as mixtures.

These dyes may be monochromophoric dyes (i.e. comprising only one dye) or polychromophoric, preferably dichromophoric or trichromophoric, dyes; the chromophores may be identical or different, and from the same chemical family or otherwise. It should be noted that a polychromophoric dye comprises several radicals each derived from a molecule that absorbs in the visible region between 400 and 800 nm. Furthermore, this absorbance of the dye does not require any prior oxidation thereof, or combination with any other chemical species.

In the case of polychromophoric dyes, the chromophores are connected together by means of at least one linker, which may be cationic or non-cationic.

Among the benzene direct dyes that may be used according to the invention, mention may be made in a non-limiting manner of the following compounds:
1,4-diamino-2-nitrobenzene,
1-amino-2 nitro-4-β-hydroxyethylaminobenzene,
1-amino-2 nitro-4-bis-(β-hydroxyethyl)aminobenzene,
1,4-bis(β-hydroxyethylamino)-2-nitrobenzene,
1-β-hydroxyethylamino-2-nitro-4-bis(β-hydroxyethylamino)benzene,
1-β-hydroxyethylamino-2-nitro-4-aminobenzene,
1-β-hydroxyethylamino-2-nitro-4-(ethyl)(β-hydroxyethyl)aminobenzene,
1-amino-3-methyl-4-β-hydroxyethylamino-6-nitrobenzene,
1-amino-2-nitro-4-β-hydroxyethylamino-5-chlorobenzene,
1,2-diamino-4-nitrobenzene,
1-amino-2-β-hydroxyethylamino-5-nitrobenzene,
1,2-bis(β-hydroxyethylamino)-4-nitrobenzene,
1-amino-2-tris(hydroxymethyl)methylamino-5-nitrobenzene,
1-hydroxy-2-amino-5-nitrobenzene,
1-hydroxy-2-amino-4-nitrobenzene,
1-hydroxy-3-nitro-4-aminobenzene,
1-hydroxy-2-amino-4,6-dinitrobenzene,
1-β-hydroxyethyloxy-2-β-hydroxyethylamino-5-nitrobenzene,
1-methoxy-2-β-hydroxyethylamino-5-nitrobenzene,
1-β-hydroxyethyloxy-3-methylamino-4-nitrobenzene,
1-β,γ-dihydroxypropyloxy-3-methylamino-4-nitrobenzene,
1-β-hydroxyethylamino-4-β,γ-dihydroxypropyloxy-2-nitrobenzene,
1-β,γ-dihydroxypropylamino-4-trifluoromethyl-2-nitrobenzene,
1-β-hydroxyethylamino-4-trifluoromethyl-2-nitrobenzene,
1-β-hydroxyethylamino-3-methyl-2-nitrobenzene,
1-β-aminoethylamino-5-methoxy-2-nitrobenzene,
1-hydroxy-2-chloro-6-ethylamino-4-nitrobenzene,
1-hydroxy-2-chloro-6-amino-4-nitrobenzene,
1-hydroxy-6-bis(β-hydroxyethyl)amino-3-nitrobenzene,
1-β-hydroxyethylamino-2-nitrobenzene,
1-hydroxy-4-β-hydroxyethylamino-3-nitrobenzene.

Among the azo, azomethine, methine and tetraazapentamethine direct dyes that may be used according to the invention, mention may be made of the cationic dyes described in patent applications WO 95/15144, WO 95/01772 and EP 714 954; FR 2 189 006, FR 2 285 851, FR 2 140 205, EP 1 378 544 and EP 1 674 073.

Among these, mention may also be made of the following compounds:

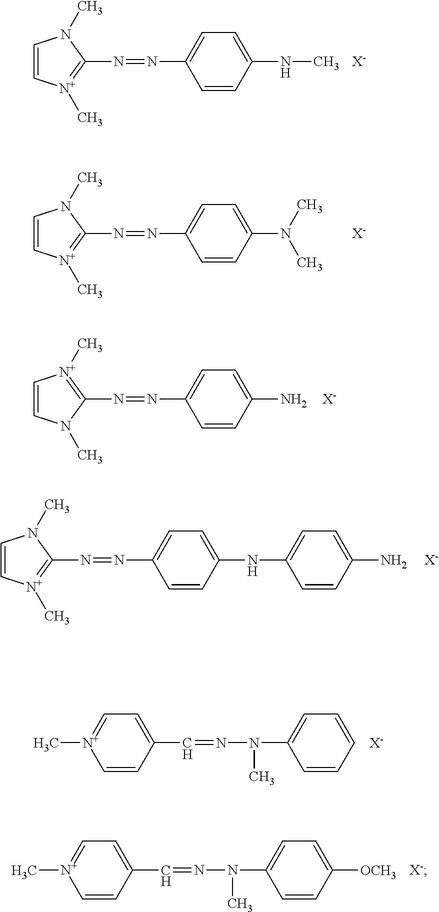

Among the azo direct dyes that may also be mentioned are the following dyes, described in the Colour Index International, 3rd edition:
Disperse Red 17
Basic Red 22
Basic Red 76
Basic Yellow 57
Basic Brown 16
Basic Brown 17
Disperse Black 9.

Mention may also be made of 1-(4′-aminodiphenylazo)-2-methyl-4-bis(β-hydroxyethyl)aminobenzene.

Among the quinone direct dyes that may be mentioned are the following dyes:
Disperse Red 15
Solvent Violet 13
Disperse Violet 1
Disperse Violet 4
Disperse Blue 1
Disperse Violet 8
Disperse Blue 3
Disperse Red 11
Disperse Blue 7
Basic Blue 22
Disperse Violet 15
Basic Blue 99
   and also the following compounds:
1-N-methylmorpholiniumpropylamino-4-hydroxyanthraquinone
1-aminopropylamino-4-methylaminoanthraquinone;
1-aminopropylaminoanthraquinone;
5-β-hydroxyethyl-1,4-diaminoanthraquinone;
2-aminoethylaminoanthraquinone; and
1,4-bis(β,γ-dihydroxypropylamino)anthraquinone.

Among the azine dyes that may be mentioned are the following compounds:
Basic Blue 17
Basic Red 2.

Among the triarylmethane dyes that may be used according to the invention, mention may be made of the following compounds:
Basic Green 1
Basic Violet 3
Basic Violet 14
Basic Blue 7
Basic Blue 26.

Among the indoamine dyes that can be used according to the invention, mention may be made of the following compounds:
2-β-hydroxyethlyamino-5-[bis(β-4′-hydroxyethyl)amino]anilino-1,4-benzoquinone;
2-β-hydroxyethylamino-5-(2′-methoxy-4′-amino)anilino-1,4-benzoquinone;
3-N-(2′-chloro-4′-hydroxy)phenylacetylamino-6-methoxy-1,4-benzoquinoneimine
3-N-(3′-chloro-4′-methylamino)phenylureido-6-methyl-1,4-benzoquinoneimine; and
3-[4′-N-(ethyl,carbamylmethyl)amino]phenylureido-6-methyl-1,4-benzoquinoneimine.

Among the dyes of tetraazapentamethine type that may be used according to the invention, mention may be made of the following compounds given in the table below, An being defined as previously:

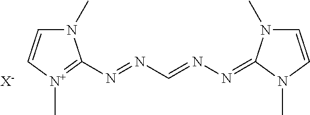

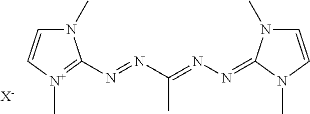

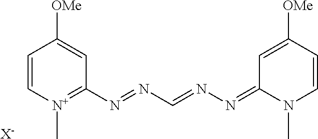 [SMILES: "X-" ; COc1cc[n+](C)c(/N=N/C=N/N=c2\cc(OC)ccn2C)c1]

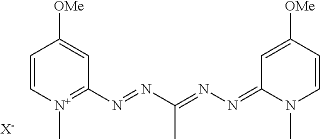 [SMILES: "X-" ; COc1cc[n+](C)c(/N=N/C(C)=N/N=c2\cc(OC)ccn2C)c1]

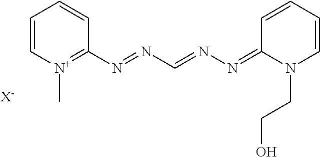 [SMILES: "X-" ; C[n+]1ccccc1/N=N/C=N/N=c1\ccccn1CCO]

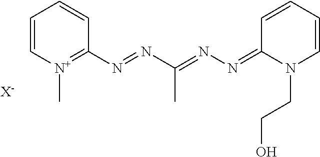 [SMILES: "X-" ; CC(/N=N/c1cccc[n+]1C)=N\N=c1/ccccn1CCO]

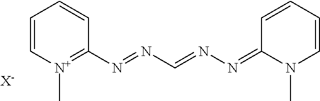

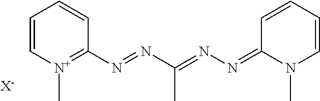

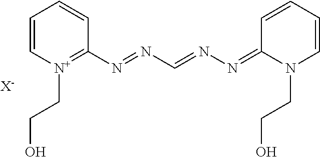

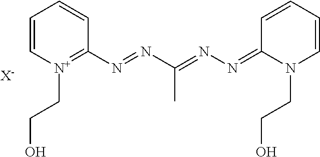

X⁻ represents an anion preferably chosen from chloride, iodide, methyl sulfate, ethyl sulfate, acetate and perchlorate.

Among the polychromophoric dyes, reference may be made more particularly to patent applications EP 1 637 566, EP 1 619 221, EP 1 634 926, EP 1 619 220, EP 1 672 033, EP 1 671 954, EP 1 671 955, EP 1 679 312, EP 1 671 951, EP 167 952, EP 167 971, WO 06/063 866, WO 06/063 867, WO 06/063 868, WO 06/063 869, EP 1 408 919, EP 1 377 264, EP 1 377 262, EP 1 377 261, EP 1 377 263, EP 1 399 425, EP 1 399 117, EP 1 416 909, EP 1 399 116 and EP 1 671 560.

It is also possible to use the cationic direct dyes mentioned in patent applications: EP 1 006 153, which describes dyes comprising two chromophores of anthraquinone type connected via a cationic linker; EP 1 433 472, EP 1 433 474, EP 1 433 471 and EP 1 433 473, which describe identical or different dichromophoric dyes, connected via a cationic or non-cationic linker, and also EP 6 291 333, which especially describes dyes comprising three chromophores, one of them being an anthraquinone chromophore, to which are attached two chromophores of azo or diazacarbocyanin type or an isomer thereof.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin, orceins, haematin, haematoxylin, brasilein and brasilin. Extracts or decoctions containing these natural dyes and in particular henna-based poultices or extracts, may also be used.

When they are present, the direct dye(s) more particularly represent from 0.0001% to 10% by weight and preferably from 0.005% to 5% by weight relative to the total weight of composition A and/or of composition (A1).

### Oxidation Dyes

The oxidation dyes are generally chosen from oxidation bases optionally combined with one or more couplers.

By way of example, the oxidation bases are chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylene-diamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetyl-amino-ethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis-(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethyl-phenol, 4-amino-2-(β-hydroxy-ethyl-aminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases that are useful in the present invention are the 3-aminopyrazolo[1,5-a]pyridine oxidation bases or addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and the addition salts thereof.

The pyrimidine derivatives include the compounds described, for example, in the patents DE 2359399; JP 88-169571; JP 05-63124; EP 0770375 or patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives, mention may be made of the compounds described in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triamino-pyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof. 4,5-Diamino-1-(β-methoxyethyl)pyrazole may also be used.

A heterocyclic base that may also be mentioned is 2,3-diamino-6,7-dihydro-1H,5H-pyrazolo[1,2-a]pyrazol-1-one or a salt thereof.

The compositions according to the invention may optionally comprise one or more couplers advantageously selected from those conventionally used for the dyeing of keratin fibres.

Among these couplers, mention may be made especially of meta-phenylenediamines, meta-aminophenols, meta-diphenols, naphthalene-based couplers and heterocyclic couplers, and also the addition salts thereof.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxy-benzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzo-morpholine, 3,5-diamino-2,6-dimethoxypyridine, 1-N-(8-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl-[3,2-c]-1,2,4-triazole and 6-methylpyrazolo[1,5-a]benzimidazole, the addition salts thereof with an acid, and mixtures thereof.

In general, the addition salts of the oxidation bases and couplers that may be used in the context of the invention are especially selected from the addition salts with an acid such as the hydrochlorides, hydrobromides, sulfates, citrates, succinates, tartrates, lactates, tosylates, benzenesulfonates, phosphates and acetates.

The oxidation base(s), if they are present, each advantageously represent from 0.0001% to 10% by weight and preferably from 0.005% to 5% by weight relative to the total weight of the dye composition (A) or (A1).

The coupler(s), if they are present, each advantageously represent from 0.0001% to 10% by weight and preferably from 0.005% to 5% by weight relative to the total weight of the dye composition (A) or (A1).

The dye composition (A) in accordance with the invention may also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, antioxidants, penetrants, sequestrants, fragrances, buffers, dispersants, conditioning agents, for instance silicones, film-forming agents, preserving agents and opacifiers.

The dye composition according to the invention may be in various forms, such as in the form of liquids, creams or gels, or in any other form that is suitable for dyeing keratin fibres, and especially human hair.

The nature of the oxidizing agent used in the lightening direct dyeing (direct dyeing with an oxidizing agent) or in the oxidation dyeing is not critical.

The oxidizing agent is preferably chosen from the group formed by hydrogen peroxide, urea peroxide, alkali metal bromates or ferricyanides, peroxygenated salts, for instance persulfates, perborates, peracids and precursors thereof and alkali metal or alkaline-earth metal percarbonates. One or more redox enzymes such as laccases, peroxidases and 2-electron oxidoreductases (such as uricase), optionally in the presence of the respective donor or cofactor thereof, may also be used as oxidizing agent.

This oxidizing agent is advantageously formed by hydrogen peroxide, especially as an aqueous solution (aqueous hydrogen peroxide solution), the titre of which may range more particularly from 1 to 40 volumes and more preferably still from 5 to 40 volumes.

### II/Skin Uses

### 1) Skin Colouring

### a) Self-Tanning Agents

According to one particular form of the invention, it is possible to apply, in a first stage, a composition (A) comprising, in a cosmetically acceptable medium, at least one self-tanning agent, and then, in a second stage, to apply to the skin a coating formed in situ from a composition (B) as defined previously comprising, in a cosmetically acceptable medium, at least the copolymer containing polyamide blocks and polyether blocks in solution or in dispersion, and at least one organic solvent.

The invention also relates to a process for colouring the skin, which consists in applying to the skin a composition (A) comprising, in a cosmetically acceptable medium, at least one self-tanning agent, and then, in a second stage, in applying to the skin a coating formed in situ from a liquid composition (B) as defined previously comprising, in a cosmetically acceptable medium, at least one copolymer containing polyamide blocks and polyether blocks in solution or in dispersion, and at least one organic solvent.

The self-tanning agents are generally chosen from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in patent application FR 2 466 492 and WO 97/35842, dihydroxyacetone (DHA) and 4,4-dihydroxypyrazolin-5-one derivatives as described in patent application EP 903 342. DHA will preferably be used.

The DHA may be used in free and/or encapsulated form, for example in lipid vesicles such as liposomes, especially described in patent application WO 97/25970.

In general, the self-tanning agent is present in an amount ranging from 0.01% to 20% by weight and preferably in an amount of between 0.1% and 10% of the total weight of the composition.

Other dyes that allow modification of the colour produced by the self-tanning agent may also be used.

These dyes may be chosen from synthetic or natural direct dyes.

These dyes may be chosen, for example, from red or orange dyes of the fluorane type such as those described in patent application FR 2 840 806. Mention may be made, for example, of the following dyes:
tetrabromofluoresceine or eosin known under the CTFA name: CI-45380 or Red 21
phloxin B known under the CTFA name: CI-45410 or Red 27
diiodofluoresceine known under the CTFA name: CI-45425 or Orange 10;
dibromofluoresceine known under the CTFA name: CI-45370 or Orange 5;
the sodium salt of tetrabromofluoresceine known under the CTFA name: CI-45380 (Na salt) or Red 22;
the sodium salt of phloxin B known under the CTFA name: CI-45410 (Na salt) or Red 28;
the sodium salt of diiodofluoresceine known under the CTFA name: CI-45425 (Na salt) or Orange 11;
erythrosine known under the CTFA name: CI-45430 or Acid Red 51.
phloxin known under the CTFA name: CI-45405 or Acid Red 98.

These dyes may also be chosen from anthraquinones, caramel, carmine, carbon black, azulene blues, methoxalene, trioxalene, guajazulene, chamuzulene, rose Bengal, eosin 10B, cyanosin and daphinin.

These dyes may also be chosen from indole derivatives, for instance the monohydroxyindoles as described in patent FR 2 651 126 (i.e.: 4-, 5-, 6- or 7-hydroxyindole) or the dihydroxyindoles as described in patent EP-B-0 425 324 (i.e.: 5,6-dihydroxyindole, 2-methyl-5,6-dihydroxyindole, 3-methyl-5,6-dihydroxyindole or 2,3-dimethyl-5,6-dihydroxyindole).

### b) Makeup

The invention also relates to a process for making up the skin, the lips, the nails, the eyelashes or the eyebrows, which consists in applying to the area to be made up a makeup composition (A) comprising at least one dyestuff, and then, in a second stage, in applying to the made-up area a coating formed in situ from a composition (B) as defined previously comprising, in a cosmetically acceptable medium, at least one copolymer containing polyamide blocks and polyether blocks in solution or in dispersion, and at least one organic solvent.

The makeup composition (A) may be in various forms, as a function of its intended use. The cosmetic composition may thus be in any galenical form normally used for topical application and especially in anhydrous form, in the form of an oily or aqueous solution, an oily or aqueous gel, an oil-in-water, water-in-oil, wax-in-water or water-in-wax emulsion, a multiple emulsion, or a dispersion of oil in water by means of vesicles located at the oil/water interface.

The composition may be in the form of a cast product, especially a stick in the case of a lipstick or of a lipcare product; or in the form of a cast foundation that may be anhydrous or in the form of a solid emulsion.

Composition (A) may also be in various other forms, for example a more or less viscous liquid, a gel or a paste.

Composition (A) may also be in the form of a semi-solid or a solid, for example a cake to be moistened at the time of use, so as to allow it to be broken down.

Composition (A) may constitute, inter alia, a lipstick, a liquid gloss, a lipstick paste, a face powder, a lip pencil, a solid or fluid foundation, a concealer or eye contour product, an eyeliner, a mascara, a nail varnish, an eyeshadow or a body makeup product.

### Dyestuffs

The makeup compositions according to the invention comprise at least one dyestuff.

A cosmetic makeup composition in accordance with the invention may advantageously incorporate at least one dyestuff chosen from organic or mineral dyes, especially such as the pigments or nacres conventionally used in cosmetic compositions, liposoluble or water-soluble dyes, materials with a specific optical effect, and mixtures thereof.

The term “pigments” should be understood as meaning white or coloured, mineral or organic particles that are insoluble in an aqueous solution, which are intended to colour and/or opacify the resulting film.

The pigments may be present in a proportion of from 0.1% to 40% by weight, especially from 1% to 30% by weight and in particular from 5% to 15% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

As illustrations of nacres that may be used in the context of the present invention, mention may be made of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nuantique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale 0005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

The cosmetic composition according to the invention may also comprise water-soluble or liposoluble dyes. The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice and caramel.

The makeup composition according to the invention may also contain at least one material with a specific optical effect.

This effect is different from a simple conventional hue effect, i.e. a unified and stabilized effect as produced by standard dyestuffs, for instance monochromatic pigments. For the purposes of the invention, the term “stabilized” means lacking the effect of variability of the colour with the angle of observation or in response to a temperature change.

For example, this material may be chosen from particles with a metallic tint, goniochromatic colouring agents, diffracting pigments, thermochromic agents, optical brighteners, and also fibres, especially interference fibres. Needless to say, these various materials may be combined so as to afford the simultaneous manifestation of two effects, or even of a novel effect in accordance with the invention.

The particles with a metallic tint that may be used in the invention are in particular chosen from:
particles of at least one metal and/or of at least one metal derivative,
particles comprising a monomaterial or multimaterial organic or mineral substrate, at least partially coated with at least one layer with a metallic tint comprising at least one metal and/or at least one metal derivative, and mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are preferred metals.

The term “metal derivatives” is intended to denote compounds derived from metals, especially oxides, fluorides, chlorides and sulfides.

Metallic particles include particles of aluminium, such as those sold under the names Starbrite 1200 EAC® by Silberline and Metalure® by Eckart.

Mention may also be made of metal powders of copper or of alloy mixtures such as the references 2844 sold by the company Radium Bronze, metallic pigments, for instance aluminium or bronze, such as those sold under the names Rotosafe 700 from the company Eckart, silica-coated aluminium particles sold under the name

Visionaire Bright Silver® from the company Eckart, and metal alloy particles, for instance the silica-coated bronze (alloy of copper and zinc) powders sold under the name Visionaire Bright Natural Gold® from the company Eckart.

They may also be particles comprising a glass substrate, for instance those sold by the company Nippon Sheet Glass under the name Microglass Metashine®.

The goniochromatic colouring agent may be chosen, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by the company DuPont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be pigments of silica/titanium oxide/tin oxide structure sold under the name Xirona Magic by the company Merck, pigments of silica/brown iron oxide structure sold under the name Xirona Indian Summer by the company Merck, and pigments of silica/titanium oxide/mica/tin oxide structure sold under the name Xirona Caribbean Blue by the company Merck. Mention may also be made of the Infinite Colors pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chemx and also the product sold under the name Helicone® HC by the company Wacker.

The makeup compositions of the invention may also contain fluorescers.

The fluorescers may be optical brighteners chosen, for example, from solutions of stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trade names Tinopal SOP® and Uvitex OB® from the company Ciba Geigy.

It is also possible to use as optical brighteners aqueous solutions of disodium distyrylbiphenyl disulfonate, which are available, for example, under the trade name Tinopal CBS X by the company CIBA.

### 2) Photoprotection of Keratin Materials

The coatings in accordance with the present invention may also be used for photoprotecting human keratin materials, in particular the skin and the hair.

Another subject of the invention is a process for screening out UV radiation on keratin materials, characterized in that a coating formed in situ from a composition (B) as defined previously, comprising, in a cosmetically acceptable medium, at least the copolymer containing polyamide blocks and polyether blocks in solution or in dispersion, at least one organic solvent and at least one organic UV-screening agent and/or at least one mineral UV-screening agent, is applied to the surface of the keratin material.

This type of the process especially allows uptake of the UV-screening agents onto the surface of keratin materials, and especially water-soluble screening agents, which are generally easy to remove with water. The coatings of the invention are suited to daily photoprotection due to the fact that they are resistant to water and to movements without causing any discomfort.

According to another variant of the invention, another subject of the invention is a process for screening out UV radiation on the surface of keratin materials, characterized in that the following are applied to the keratin material:
a) a composition (A) comprising, in a cosmetically acceptable medium, at least one organic UV-screening agent and/or at least one mineral UV-screening agent,
b) a coating formed in situ from a composition (B) as defined previously, comprising, in a cosmetically acceptable medium, at least the copolymer containing polyamide blocks and polyether blocks in solution or in dispersion, and at least one organic solvent.

Another subject of the invention is thus a cosmetic assembly comprising at least:
(iii) a first composition (A) as defined previously;
(iv) a second composition (B) as defined previously.

The compositions (A) may be prepared according to techniques that are well known to those skilled in the art. They may in particular be in the form of a simple or complex emulsion (O/W, W/O, O/W/O or W/O/W) such as a cream or a milk; in the form of a lotion; in the form of a stick. They may optionally be packaged as an aerosol and may be in the form of a mousse or a spray.

According to the invention, the photoprotective system present in the composition (B) may be formed from one or more hydrophilic, lipophilic or insoluble organic screening agents and/or one or more mineral pigments.

The organic UV-screening agents are chosen especially from cinnamic derivatives; anthranilates; salicylic derivatives; dibenzoylmethane derivatives, camphor derivatives; benzophenone derivatives; β,β-diphenylacrylate derivatives; triazine derivatives; benzotriazole derivatives; benzalmalonate derivatives, especially those mentioned in patent U.S. Pat. No. 5,624,663; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; α-alkyl-styrene-based dimers, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981; merocyanin derivatives such as those described in patent applications WO 04/006 878, WO 05/058 269 and WO 06/032 741; and mixtures thereof.

As examples of additional organic photoprotective agents, mention may be made of those denoted hereinbelow under their INCI name:

### Cinnamic Derivatives:

Ethylhexyl methoxycinnamate sold especially under the trade name Parsol MCX by DSM Nutritional Products,

Isopropyl methoxycinnamate,

Isoamyl methoxycinnamate sold under the trade name Neo Heliopan E 1000 by Symrise,

DEA methoxycinnamate,

Diisopropyl methylcinnamate,

Glyceryl ethylhexanoate dimethoxycinnamate

### Dibenzoylmethane Derivatives:

Butylmethoxydibenzoylmethane sold especially under the trade name Parsol 1789 by DSM,

Isopropyldibenzoylmethane.

### Para-Aminobenzoic Acid Derivatives:

PABA,

Ethyl PABA,

Ethyl Dihydroxypropyl PABA,

Ethylhexyl dimethyl PABA sold in particular under the name Escalol 507 by ISP, Glyceryl PABA,

PEG-25 PABA sold under the name Uvinul P25 by BASF,

### Salicylic Derivatives:

Homosalate sold under the name Eusolex HMS by Rona/EM Industries,

Ethylhexyl salicylate sold under the name Neo Heliopan OS by Symrise,

Dipropylene glycol salicylate sold under the name Dipsal by Scher,

TEA salicylate sold under the name Neo Heliopan TS by Symrise.

### β,β-Diphenylacrylate Derivatives:

Octocrylene sold especially under the trade name Uvinul N539 by BASF,

Etocrylene sold especially under the trade name Uvinul N35 by BASF,

### Benzophenone Derivatives:

Benzophenone-1 sold under the trade name Uvinul 400 by BASF,

Benzophenone-2 sold under the trade name Uvinul D50 by BASF,

Benzophenone-3 or Oxybenzone sold under the trade name Uvinul M40 by BASF,

Benzophenone-4 sold under the trade name Uvinul MS40 by BASF,

Benzophenone-5,

Benzophenone-6 sold under the trade name Helisorb 11 by Norquay,

Benzophenone-8 sold under the trade name Spectra-Sorb UV-24 by American Cyanamid,

Benzophenone-9 sold under the trade name Uvinul DS-49 by BASF,

Benzophenone-12,

n-hexyl 2-(4-diethylamino-2-hydroxybenzoyl)benzoate sold under the trade name

Uvinul A+, or in the form of a mixture with octyl methoxycinnamate under the trade name Uvinul A+B by the company BASF.

### Benzylidenecamphor Derivatives:

3-Benzylidenecamphor manufactured under the name Mexoryl SD by Chimex,

4-Methylbenzylidenecamphor sold under the name Eusolex 6300 by Merck,

Benzylidenecamphorsulfonic acid manufactured under the name Mexoryl SL by Chimex,

Camphor benzalkonium methosulfate manufactured under the name Mexoryl SO by Chimex,

Terephthalylidenedicamphorsulfonic acid manufactured under the name Mexoryl SX by Chimex,

Polyacrylamidomethylbenzylidenecamphor manufactured under the name Mexoryl SW by Chimex,

### Phenylbenzimidazole Derivatives:

Phenylbenzimidazolesulfonic acid sold in particular under the trade name Eusolex 232 by Merck,

Disodium phenyl dibenzimidazole tetrasulfonate sold under the trade name Neo Heliopan AP by Symrise.

### Phenylbenzotriazole Derivatives:

Drometrizole trisiloxane sold under the name Silatrizole by Rhodia Chimie, Methylenebis(benzotriazolyl)tetramethylbutylphenol sold in solid form under the trade name Mixxim BB/100 by Fairmount Chemical, or in micronized form as an aqueous dispersion under the trade name Tinosorb M by Ciba Specialty Chemicals.

### Triazine Derivatives:

Bis(ethylhexyloxyphenol)methoxyphenyltriazine sold under the trade name Tinosorb S by Ciba Geigy,

Ethylhexyltriazone sold in particular under the trade name Uvinul T150 by BASF,

Diethylhexylbutamidotriazone sold under the trade name Uvasorb HEB by Sigma 3V,

2,4,6-tris(dineopentyl 4′-aminobenzalmalonate)-s-triazine

2,4,6-tris(diisobutyl 4′-aminobenzalmalonate)-s-triazine,

2,4-bis(dineopentyl 4′-aminobenzalmalonate)-6-(n-butyl 4′-aminobenzoate)-s-triazine, the symmetrical triazine screening agents described in patent U.S. Pat. No. 6,225,467, patent application WO 2004/085 412 (see compounds 6 and 9) or the document Symmetrical Triazine Derivatives IP.COM Journal, IP.COM INC West Henrietta, N.Y., US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which is also mentioned in patent applications WO 06/035 000, WO 06/034 982, WO 06/034 991, WO 06/035 007, WO 2006/034 992 and WO 2006/034 985.

### Anthranilic Derivatives:

Menthyl anthranilate sold under the trade name Neo Heliopan MA by Symrise,

### Imidazoline Derivatives:

Ethylhexyldimethoxybenzylidenedioxoimidazoline propionate,

### Benzalmalonate Derivatives:

Polyorganosiloxane containing benzalmalonate functions, for instance Polysilicone-15, sold under the trade name Parsol SLX by DSM Nutritional Products.

### 4,4-Diarylbutadiene Derivatives:

1,1-Dicarboxy(2,2′-dimethylpropyl)-4,4-diphenyl-butadiene,

### Benzoxazole Derivatives:

2,4-bis[5-1(dimethylpropyl)benzoxazol-2-yl-(4-phenyl)imino]-6-(2-ethylhexyl)imino-1,3,5-triazine, sold under the name of Uvasorb K2A by Sigma 3V, and mixtures thereof.

The preferential organic screening agents are chosen from:

Ethylhexyl methoxycinnamate,

Ethylhexyl salicylate,

Homosalate,

Butylmethoxydibenzoylmethane,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)-benzoate,

4-Methylbenzylidenecamphor,

Terephthalylidenedicamphorsulfonic acid,

Disodium phenyldibenzimidazoletetrasulfonate,

Methylenebis(benzotriazolyl)tetramethylbutyl-phenol,

Bis(ethylhexyloxyphenol)methoxyphenyltriazine,

Ethylhexyl triazone,

Diethylhexylbutamidotriazone,

2,4,6-Tris(dineopentyl 4′-aminobenzalmalonate)-s-triazine,

2,4,6-Tris(diisobutyl 4′-aminobenzalmalonate)-s-triazine,

2,4-Bis(dineopentyl 4′-aminobenzalmalonate)-6-(n-butyl 4′-aminobenzoate)-s-triazine,

2,4,6-Tris(biphenyl-4-yl)-1,3,5-triazine,

2,4,6-Tris(terphenyl)-1,3,5-triazine,

Drometrizole trisiloxane,

Polysilicone-15,

1,1-Dicarboxy(2,2′-dimethylpropyl)-4,4-diphenyl-butadiene, 2,4-Bis[5-1(dimethylpropyl)benzoxazol-2-yl-(4-phenyl)imino]-6-(2-ethylhexypimino-1,3,5-triazine,

and mixtures thereof.

The mineral UV-screening agents used in accordance with the present invention are metal oxide pigments. More preferentially, the mineral UV-screening agents of the invention are metal oxide pigments with a mean elemental particle size of less than or equal to 500 nm, more preferentially between 5 nm and 500 nm and even more preferentially between 10 nm and 100 nm, and preferentially between 15 and 50 nm.

They may be chosen especially from titanium oxide, zinc oxide, iron oxide, zirconium oxide and cerium oxide, or mixtures thereof.

Such coated or uncoated metal oxide pigments are described in particular in patent application EP-A-0 518 773. Commercial pigments that may be mentioned include the products sold by the companies Kemira, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium) of polyethylene, silicones, proteins (collagen, elastin), alkanolamines, silicon oxides, metal oxides or sodium hexametaphosphate.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca and Tioveil from the company Tioxide,
with alumina, such as the products Tipaque TTO-55 (B) and Tipaque TTO-55 (A) from the company Ishihara and UVT 14/4 from the company Kemira,
with alumina and aluminium stearate, such as the product Microtitanium Dioxide MT 100 T, MT 100 TX, MT 100 Z and MT-01 from the company Tayca, the products Solaveil CT-10 W and Solaveil CT 100 from the company Uniqema and the product Eusolex T-AVO from the company Merck,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.
TiO₂ treated with octyltrimethylsilane, sold under the trade name T 805 by the company Degussa Silices,
TiO₂ treated with a polydimethylsiloxane, sold under the trade name 70250 Cardre UF TiO2SI3 by the company Cardre,
anatase/rutile TiO₂ treated with a polydimethylhydrogenosiloxane, sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wacker under the name Transparent titanium oxide PW, by the company Miyoshi Kasei under the name UFTR, by the company Tomen under the name ITS and by the company Tioxide under the name Tioveil AQ.

The uncoated zinc oxide pigments are, for example:
those sold under the name Z-Cote by the company Sunsmart;
those sold under the name Nanox by the company Elementis;
those sold under the name Nanogard WCD 2025 by the company Nanophase Technologies.

The coated zinc oxide pigments are, for example:
those sold under the name Zinc Oxide CS-5 by the company Toshibi (ZnO coated with polymethylhydrogenosiloxane);
those sold under the name Nanogard Zinc Oxide FN by the company Nanophase Technologies (as a 40% dispersion in Finsolv TN, C12-C15 alkyl benzoate);
those sold under the name Daitopersion ZN-30 and Daitopersion ZN-50 by the company Daito (dispersions in cyclopolymethylsiloxane/oxyethylenated polydimethyl-siloxane, containing 30% or 50% of nanozinc oxides coated with silica and polymethylhydrogenosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol Z100 by the company ISP (alumina-treated ZnO dispersed in an ethylhexyl methoxycinnamate/PVP-hexadecene/methicone copolymer mixture);
those sold under the name Fuji ZnO-SMS-10 by the company Fuji Pigment (ZnO coated with silica and polymethylsilsesquioxane);
those sold under the name Nanox Gel TN by the company Elementis (ZnO dispersed at a concentration of 55% in C12-C15 alkyl benzoate with hydroxystearic acid polycondensate).

The uncoated cerium oxide pigments are sold under the name Colloidal Cerium Oxide by the company Rhone-Poulenc.

The uncoated iron oxide nanopigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220.

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, especially of titanium dioxide and of cerium dioxide, including the silica-coated equal-weight mixture of titanium dioxide and of cerium dioxide, sold by the company Ikeda under the name Sunveil A, and also the alumina, silica and silicone-coated mixture of titanium dioxide and of zinc dioxide, such as the product M 261 sold by the company Kemira, or the alumina, silica and glycerol-coated mixture of titanium dioxide and of zinc dioxide, such as the product M 211 sold by the company Kemira.

According to the invention, coated or uncoated titanium oxide pigments are particularly preferred.

The screening agents are preferably present in the compositions according to the invention in a content ranging from 0.1% to 40% by weight and in particular from 5% to 25% by weight relative to the total weight of the composition.

### 3) Treatment of Perspiration and Body Odour

The coatings in accordance with the present invention may also be used for treating human perspiration and the body odour resulting therefrom, especially in the armpits. The coatings of the invention, by virtue of their mechanical properties, withstand the inevitable friction in the region of the armpits.

Another subject of the invention is a process for treating human perspiration and the body odour resulting therefrom, characterized in that a coating formed in situ from a composition (B) as defined previously, comprising, in a cosmetically acceptable medium, at least the copolymer containing polyamide blocks and polyether blocks in solution or in dispersion, and at least one organic solvent is applied to the area of the body to be treated.

Composition (B) according to the invention may also contain an agent for treating perspiration and/or a deodorant active agent.

The term “agent for treating perspiration” means any substance which, by itself, has the effect of reducing the sensation on the skin of moisture associated with human sweat, or of masking human sweat.

The antiperspirant salts or complexes in accordance with the invention are generally chosen from aluminium and/or zirconium salts or complexes. They are preferably chosen from aluminium halohydrates; aluminium zirconium halohydrates, complexes of zirconium hydroxychloride and of aluminium hydroxychloride with or without an amino acid, such as those described in patent U.S. Pat. No. 3,792,068.

Among the aluminium salts, mention may be made in particular of aluminium chlorohydrate in activated or unactivated form, aluminium chlorohydrex, the aluminium chlorohydrex-polyethylene glycol complex, the aluminium chlorohydrex-propylene glycol complex, aluminium dichlorohydrate, the aluminium dichlorohydrex-polyethylene glycol complex, the aluminium dichlorohydrex-propylene glycol complex, aluminium sesquichlorohydrate,the aluminium sesquichlorohydrex-polyethylene glycol complex, the aluminium sesquichlorohydrex-propylene glycol complex, aluminium sulfate buffered with sodium aluminium lactate.

Among the aluminium-zirconium salts, mention may be made in particular of aluminium zirconium octachlorohydrate, aluminium zirconium pentachlorohydrate, aluminium zirconium tetrachlorohydrate and aluminium zirconium trichlorohydrate.

The complexes of zirconium hydroxychloride and of aluminium hydroxychloride with an amino acid are generally known as ZAG (when the amino acid is glycine). Among these products, mention may be made of the complexes aluminium zirconium octachlorohydrex glycine, aluminium zirconium pentachlorohydrex glycine, aluminium zirconium tetrachlorohydrex glycine and aluminium zirconium trichlorohydrex glycine.

The antiperspirant active agents may be present in the composition according to the invention in a proportion from about 0.5% to 25% by weight relative to the total weight of the composition.

The compositions according to the invention may also contain one or more additional deodorant active agents.

The term “deodorant active agent” refers to any substance that is capable of masking, absorbing, improving and/or reducing the unpleasant odour resulting from the decomposition of human sweat by bacteria.

The deodorant active agents may be bacteriostatic agents or bactericides that act on underarm odour microorganisms, such as 2,4,4′-trichloro-2′-hydroxydiphenyl ether (®Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (®Triclocarban) or 3,7,11-trimethyldodeca-2,5,10-trienol®Farnesol); quaternary ammonium salts such as cetyltrimethylammonium salts, cetylpyridinium salts, DPTA (1,3-diaminopropanetetraacetic acid), 1,2-decanediol (Symclariol from the company Symrise), biguanide derivatives, for instance polyhexamethylene biguanide salts, chlorhexidine and salts thereof; 4-phenyl-4,4-dimethyl-2-butanol (Symdeo MPP from Symrise).

Among the additional deodorant active agents, mention may also be made of: —zinc salts, for instance zinc salicylate, zinc gluconate, zinc pidolate; zinc sulfate, zinc chloride, zinc lactate, zinc phenolsulfonate; zinc ricinoleate;
sodium bicarbonate;
salicylic acid and derivatives thereof such as 5-n-octanoylsalicylic acid;
silver zeolites or silver-free zeolites;
alum.

The deodorant active agents may be present in the composition according to the invention in a proportion from about 0.01% to 5% by weight relative to the total weight of the composition.

Composition (B) according to the invention may also contain powders, such as spherical particles or lamellar particles.

### Spherical Particles

The spherical particles used according to the invention have or substantially have the shape of a sphere and may be hollow or solid. Advantageously, the particles of the invention have a particle size (number-average diameter) ranging from 0.1 μm to 250 μm, better still ranging from 1 μm to 150 μm and better still from 10 μm to 100 μm.

As spherical particles that may be used in the composition of the invention, examples that may be mentioned include silica powder; polyamide particles and especially Nylon 12, for instance the product sold under the name Orgasol by the company Atochem; polyethylene powders; microspheres based on acrylic copolymers, such as those made of ethylene glycol dimethacrylate/lauryl methacrylate copolymer sold by the company Dow Corning under the name Polytrap; expanded powders such as hollow microspheres, and especially the microspheres sold under the name ExpanceI by the company Kemanord Plast or under the name Micropearl F 80 ED by the company Matsumoto; powders of natural organic materials such as corn starch, wheat starch or rice starch, that may or may not be crosslinked, starch powders crosslinked with octenylsuccinate anhydride, sold under the name Dry-Flo by the company National Starch; silicone resin microbeads, in particular silsesquioxane powders described especially in patent EP 293 795, such as those sold under the name Tospearl by the company Toshiba Silicone; and mixtures thereof.

### Lamellar Particles

As indicated above, lamellar particles are particles of parallelepipedal shape (rectangular or square surface), discoid shape (circular surface) or ellipsoid shape (oval surface), characterized by three dimensions: a length, a width and a height. When the shape is circular, the length and the width are identical and correspond to the diameter of a disk, whereas the height corresponds to the thickness of the disc. When the surface is oval, the length and the width correspond, respectively, to the large axis and the small axis of an ellipse and the height corresponds to the thickness of the elliptic disc formed by the platelet. When it is a parallelepiped, the length and the which may be of identical or different dimensions: when they are of the same dimension, the shape of the surface of the parallelepiped is a square; in the contrary case, the shape is rectangular. As regards the height, it corresponds to the thickness of the parallelepiped.

The length of the lamellar particles used according to the invention preferably ranges from 0.01 to 100 μm, better still from 0.1 to 50 μm and even better still from 1 to 50 μm. The width of these platelets preferably ranges from 0.01 to 100 μm, better still from 0.1 to 50 μm and even better still from 1 to 10 μm. The height (thickness) of these platelets preferably ranges from 0.1 nm to 1 μm (0.1 to 1000 nm), better still from 1 nm to 600 nm and even better still from 1 nm to 500 nm.

As lamellar particles that may be used in the composition of the invention, mention may be made of lamellar silicates.

Lamellar silicates that may be mentioned include clays, talcs, micas, nacres and perlites, and mixtures thereof.

Clays are mixed silicates of natural or synthetic origin containing several (two or more) types of cations chosen from alkali metals (for example Na, Li, K) or alkaline-earth metals (for example Be, Mg, Ca), transition metals and aluminium.

As clays that may be used in the invention, examples that may be mentioned include sodium magnesium silicate (CTFA name: Sodium magnesium silicate), clays of the kaolin family, such as kaolin or kaolinite, dickite and nacrite; clays of the halloysite, dombassite, antigorite, benthierine or pyrophyllite family; montmorillonites; beidellite; vermiculites; stevensite; hectorites; saponites; chlorites; sepiolite; smectite, and also these clays chemically modified, for example, with acrylic acids, polysaccharides (for example carboxymethylcellulose) or organic cations, and mixtures thereof.

Talcs are hydrated magnesium silicates usually comprising aluminium silicate. The crystal structure of talc consists of repeated layers of a sandwich of brucite between layers of silica.

Micas are aluminium silicates optionally comprising iron and/or alkali metals. They have the property of being able to divide into thin layers (about 1 μm). The generally range in size from 5 to 150 μm, preferably from 10 to 100 μm and better still from 10 to 60 μm or the largest dimension (length), and a height (thickness) of from 0.1 to 0.5 μm. Micas that may be mentioned include phlogopite, muscovite, fluorophlogopite and vermiculite, and mixtures thereof. Mention may also be made of micaceous clays such as illite.

The term “nacres” should be understood as meaning iridescent particles, produced especially by certain molluscs in their shell or else synthesized, which serve to modify the texture of the composition and also the matt/gloss effect. Nacres are generally micas that are surface-treated to obtain this iridescent effect. Among the nacres that may be used in the invention, mention may be made, for example, of micas coated with titanium oxide, with iron oxide, with natural pigment and/or with bismuth oxychloride, such as coloured or uncoloured titanium oxide-mica (or titanium-mica), and mixtures thereof.

Among the lamellar silicates, mention may also be made of perlites and preferably expanded perlites.

According to another variant, a coating formed in situ from a composition (B) as defined previously, comprising, in a cosmetically acceptable medium, at least the copolymer containing polyamide blocks and polyether blocks in solution or in dispersion, and at least one organic solvent, may be applied in a first step to the area of the body to be treated, and, in a second step, a composition (A) comprising, in a cosmetically acceptable medium, at least one agent for treating perspiration, may be applied; the order of application being irrelevant.

Another subject of the invention is thus a cosmetic assembly comprising at least:

(i) a first composition (A) as defined previously;
(ii) a second composition (B) as defined previously.

### 4) Skin Fragrancing

A subject of the present invention is also a process for fragrancing a keratin material, characterized in that a coating formed in situ from a liquid composition comprising, in a cosmetically acceptable medium, at least one polyamide-polyether in solution or in dispersion, at least one organic solvent and at least one fragrancing substance is applied to the said keratin material.

A subject of the present invention is also a process for fragrancing a keratin material, characterized in that:

1) at least one composition (A) comprising, in a cosmetically acceptable medium, at least one fragrancing substance is applied to the said keratin material;
2) the said keratin materials are optionally partially or totally dried;
3) a coating formed in situ from a liquid composition (B) comprising, in a cosmetically acceptable medium, at least the copolymer containing polyamide blocks and polyether blocks in solution or in dispersion, and at least one organic solvent is applied to the said keratin material.

Another subject of the invention is thus a cosmetic assembly comprising at least:

(i) a first composition (A) as defined previously;
(ii) a second composition (B) as defined previously.

According to one particular form of this fragrancing process, the liquid composition (B) comprises, in the cosmetically acceptable medium, at least one polyamide-polyether in solution or in dispersion, at least one organic solvent and at least one agent allowing the coating to be fragmented or detached. This embodiment makes it possible to obtain slow diffusion of the fragrance, to activate the release of the fragrance by scratching or detaching the coating.

According to another particular form of the invention, the fragrancing substance(s) may be encapsulated.

Fragrancing substances are compositions especially containing the starting materials described in S. Arctander, Perfume and Flavor Chemicals (Montclair, N.J., 1969), in S. Arctander, Perfume and Flavor Materials of Natural Origin (Elizabeth, N.J., 1960) and in Flavor and Fragrance Materials—1991, Allured Publishing Co., Wheaton, Ill.

They may be natural products (essential oils, absolutes, resinoids, resins or concretes) and/or synthetic products more particularly comprising at least one aldehyde compound and/or one ketone compound, which are saturated or unsaturated, and aliphatic or cyclic.

According to the definition given in international standard ISO 9235 and adopted by the Commission of the European Pharmacopoeia, an essential oil is an odoriferous product generally of complex composition, obtained from a botanically defined plant raw material, either by steam entrainment, or by dry distillation, or via an appropriate mechanical process without heating (cold pressing). The essential oil is usually separated from the aqueous phase via a physical process that does not result in any significant change in the composition.

Essential oils are generally volatile and liquid at room temperature, which distinguishes them from “set” oils. They are more or less coloured and their density is generally less than that of water. They have a high refractive index and most of them deflect polarized light. They are liposoluble and soluble in the usual organic solvents, entrainable with steam, and very sparingly soluble in water.

Among the essential oils that may be used according to the invention, mention may be mode of those obtained from plants belonging to the following botanical families:

Abietaceae or Pinaceae: conifers

### Amaryllidaceae

### Anacardaceae

Anonaceae: ylang ylang
Apiaceae (for example Umbelliferae): dill, angelica, coriander, sea fennel, carrot, parsley

### Araceae

### Aristolochiaceae

Asteraceae: yarrow, artemisia, camomile, helichrysum

### Betulaceae

### Brassicaceae

Burseraceae: frankincense

### Carophyllaceae

### Canellaceae

Cesalpiniaceae: copaifera (copaiba balsam)

### Chenopodaceae

Cistaceae: rock rose

### Cyperaceae

### Dipterocarpaceae

Ericaceae: gaultheria (wintergreen)

### Euphorbiaceae

### Fabaceae

Geraniaceae: geranium

### Guttiferae

### Hamamelidaceae

### Hernandiaceae

Hypericaceae: St-John's wort

### Iridaceae

### Juglandaceae

Lamiaceae: thyme, oregano, monarda, savory, basil, marjorams, mints, patchouli, lavenders, sages, catnip, rosemary, hyssop, balm
Lauraceae: ravensara, sweet bay, rosewood, cinnamon, litsea
Liliaceae: garlic
Magnoliaceae: magnolia

### Malvaceae

### Meliaceae

### Monimiaceae

Moraceae: hemp, hop

### Myricaceae

Myristicaceae: nutmeg
Myrtaceae: eucalyptus, tea tree, paperbark tree, cajuput, backhousia, clove, myrtle

### Oleaceae

Piperaceae: pepper

### Pittosporaceae

Poaceae: lemon balm, lemongrass, vetiver

### Polygonaceae

### Renonculaceae

Rosaceae: roses

### Rubiaceae

Rutaceae: all citrus plants

### Salicaceae

Santalaceae: sandalwood

### Saxifragaceae

### Schisandraceae

Styracaceae: benjoin
Thymelaceae: agar wood

### Tilliaceae

Valerianaceae: valerian, spikenard
Verbenaceae: lantana, verbena

### Violaceae

Zingiberaceae: galangal, turmeric, cardamom, ginger

### Zygophyllaceae.

Mention may also be made of the essential oils extracted from flowers (lily, lavender, rose, jasmine, ylang ylang, neroli), from stems and leaves (patchouli, geranium, petitgrain), from fruit (coriander, aniseed, cumin, juniper), from fruit peel (bergamot, lemon, orange), from roots (angelica, celery, cardamom, iris, rattan palm, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benjoin, myrrh, olibanum, opopanax).

Examples of fragrancing substances are especially: α-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, 4-(4-hydroxy-4-methyl pentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranyl acetone, n-decanal, n-dodecanal, 9-decen-1-ol, phenylacetaldehyde dimethyl acetal, phenylacetaldehyde diethyl acetal, citral, citronellal, hydroxycitronellal, damascone, ionones, methylionones, isomethylionones, solanone, irones, macrocyclic ketones, musk macrolactones, ethylene brassylate, and mixtures thereof.

According to one preferred embodiment of the invention, a mixture of different fragrancing substances that generate in common a note that is pleasant to the user is used.

The fragrancing substances will preferably be chosen such that they produce notes (head, heart and base) in the following families:

citrine,
ambery,
floral,
spicy,
woody,
gourmand,
chypre,
fougere,
leathery,
musky.

The fragrancing compositions of the invention preferably contain from 1% to 40% by weight of fragrancing substance, better still from 2% to 30% by weight and in particular from 2% to 20% by weight relative to the total weight of the composition.

### 5) Skincare

According to one particular form of the invention, the cosmetic skincare process may consist in applying to the skin a coating formed in situ from a liquid composition (B) comprising, in a cosmetically acceptable medium, at least the copolymer containing polyamide blocks and polyether blocks in solution or in dispersion, at least one organic solvent and at least one skincare active agent.

According to another particular form of the invention, the cosmetic skincare process may consist in

a) applying to the said human keratin material a cosmetic composition (A) comprising at least one skincare active agent;
b) optionally rinsing and/or drying;
c) applying to the skin a coating formed in situ from a liquid composition (B) comprising, in a cosmetically acceptable medium, at least the copolymer containing polyamide blocks and polyether blocks in solution or in dispersion, and at least one organic solvent.

The composition (B) may also contain at least one skincare active agent.

Among the skincare active agents that may be used according to the invention, mention may be made of the following treating agents:

### Moisturizers or Humectants

Humectants or moisturizers that may especially be mentioned include glycerol and derivatives thereof, urea and derivatives thereof, especially Hydrovance® sold by National Starch, hyaluronic acid, AHAs, BHAs, sodium pidolate, xylitol, serine, sodium lactate, ectoin and derivatives thereof, chitosan and derivatives thereof, collagen, plankton, an extract of Imperata cylindra sold under the name Moist 24® by the company Sederma, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil sold by Nestle under the name NutraLipids®; a C-glycoside derivative such as those described in patent application WO 02/051 828 and in particular C-6-D-xylopyranoside-2-hydroxypropane in the form of a solution containing 30% by weight of active material in a water/propylene glycol mixture (60/40% by weight) such as the product sold by Chimex under the trade name Mexoryl SBBC); an oil of musk rose sold by Nestle; an extract of the microalga Prophyridium cruentum enriched with zinc, sold by Vincience under the name Algualane Zinc®; spheres of collagen and of chondroitin sulfate of marine origin (Atelocollagen) sold by the company Engelhard Lyon under the name Marine Filling Spheres; hyaluronic acid spheres such as those sold by the company Engelhard Lyon.

### Desquamating Agents

The term “desquamating agent” means any compound capable of acting:

either directly on desquamation by promoting exfoliation, such as β-hydroxy acids (BHA), in particular salicylic acid and derivatives thereof (including 5-n-octanoylsalicylic acid, also known as capryloyl salicylic acid); α-hydroxy acids (AHA), such as glycolic acid, citric acid, lactic acid, tartaric acid, malic acid or mandelic acid; 8-hexadecene-1,16-dicarboxylic acid or 9-octadecenedioic acid; urea and derivatives thereof; gentisic acid and derivatives thereof; oligofucoses; cinnamic acid; Saphora japonica extract; resveratrol, and certain jasmonic acid derivatives;
or on the enzymes involved in the desquamation or degradation of corneodesmosomes, glycosidases, stratum corneum chymotryptic enzyme (SCCE) or other proteases (trypsin, chymotrypsin-like). Mention may be made of aminosulfonic compounds and in particular 4-(2-hydroxyethyl)piperazine-1-propanesulfonic acid (HEPES); 2-oxothiazolidine-4-carboxylic acid (procysteine) derivatives; derivatives of α-amino acids of glycine type (as described in EP-0 852 949, and also sodium methyl glycine diacetate sold by BASF under the trade name Trilon M); honey; O-octanoyl-6-D-maltose and N-acetylglucosamine.

As other desquamating agents that may be used in the composition according to the invention, mention may be made of:
oligofructoses, EDTA and derivatives thereof, laminaria extracts, o-linoleyl-6D-glucose, (3-hydroxy-2-pentylcyclopentyl)acetic acid, glycerol trilactate, O-octanyl-6′-D-maltose, S-carboxymethylcysteine, siliceous derivatives of salicylate such as those described in patent EP 0 796 861, oligofucases such as those described in patent EP 0 218 200, 5-acyl salicylic acid salts, active agents with effects on transglutaminase, as in patent EP 0 899 330,
extract of the flowers of ficus Opuntia indica (Exfolactive® from Silab),
8-hexadecene-1,16-dicarboxylic acid,
esters of glucose and of vitamin F, and
mixtures thereof.

### Agents for Improving the Barrier Function

As agents for improving the barrier function, mention may be made especially of arginine, an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefossé, sphingosines, for instance salicyloyl sphingosine sold under the name Phytosphingosine® SLC by the company Degussa, a mixture of xylitol, polyxylityl glycoside and xylitan, for instance Aquaxyl® from SEPPIC, extracts of Solanacea plants, for instance Lipidessence® from Coletica, omega-3 unsaturated oils such as musk rose oils, and mixtures thereof.

Mention may also be made especially of ceramides or derivatives thereof, in particular ceramides of type 2 (for instance N-oleoyldihydrosphingosine), of type 3 (for instance stearoyl-4-hydroxysphinganine, as the INCI name) and of type 5 (for instance N-2-hydroxypalmitoyldihydrosphingosine, having the INCI name: hydroxypalmitoyl sphinganine), sphingoid-based compounds, glycosphingolipids, phospholipids, cholesterol and derivatives thereof, phytosterols, essential fatty acids, diacylglycerol, 4-chromanone and chromone derivatives, petroleum jelly, lanolin, shea butter, cocoa butter, lanolin and PCA salts.

### Antioxidants

Mention may be made especially of tocopherol and esters thereof, in particular tocopheryl acetate; ascorbic acid and derivatives thereof, in particular magnesium ascorbyl phosphate and ascorbyl glucoside; ferulic acid; serine; ellagic acid, polyphenols, tannins, tannic acid, epigallocatechins and natural extracts containing them, anthocyans, rosemary extracts, olive leaf extracts, for instance those from the company Silab, green tea extracts, resveratrol and derivatives thereof, ergothioneine, N-acetylcysteine, an extract of the brown alga Pelvetia caniculata, for instance Pelvetiane® from Secma, chlorogenic acid, biotin, chelating agents, such as BHT and BHA, N,N′-bis(3,4,5-trimethoxybenzyl)ethylenediamine and salts thereof; idebenone, plant extracts, for instance Pronalen Bioprotect™ from the company Provital; coenzyme Q10, bioflavonoids, SODs, phytanetriol, lignans, melatonin, pidolates, glutathione, caprylyl glycol, phloretin, Totarol™ or extract of Podocarpus totara containing Totarol (totara-8,11,13-trienol or 2-phenanthrenol, 4b,5,6,7,8,8a,9,10-octahydro-4-b,8,8-trimethyl-1-(1-methylethyl)-; a jasmine extract such as the product sold by Silab under the name Helisun®; hesperitin laurate such as Flavagrum PEG® from the company Engelhard Lyon; an extract of Paeonia suffruticosa root, such as the product sold by the company Ichimaru Pharcos under the name Botanpi Liquid B®, an extract of lychee such as the extract of lychee pericarp sold by the company Cognis under the name Litchiderm LS 9704®, an extract of pomegranate fruit (Punica granatum), such as the product sold by the company Draco Natural Products.

Other anti-ageing agents that may be mentioned include DHEA and derivatives thereof, boswellic acid, rosemary extracts, carotenoids (β-carotene, zeaxanthin and lutein), cysteic acid, copper derivatives and jasmonic acid.

### Depigmenting Agents

Depigmenting agents that may especially be mentioned include alpha and beta arbutin, ferulic acid, lucinol and derivatives thereof, kojic acid, resorcinol and derivatives thereof, tranexamic acid and derivatives thereof, gentisic acid, homogentisate, methyl gentisate or homogentisate, dioic acid, calcium D-pantheteine sulfonate, lipoic acid, ellagic acid, vitamin B3, linoleic acid and derivatives thereof, ceramides and homologues thereof, plant derivatives, for instance camomile, bearberry, the aloe family (vera, ferox, bardensis), mulberry or skullcap; a kiwi fruit (Actinidia chinensis) juice sold by Gattefossé, an extract of Paeonia suffruticosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, without this list being exhaustive.

### Dermo-Relaxing or Dermo-Decontracting Agents

Examples that may be mentioned include manganese gluconate and other salts, adenosine, alverine citrate and salts thereof, lysine, an extract of Iris pallida, a hexapeptide (Argeriline R from Lipotec) or sapogenins, for instance wild yam and the carbonyl amines described in patent application EP 1 484 052. Examples of sapogenins that may be mentioned include those described in patent application WO 02/47650, in particular wild yam, the diosgenin extracted especially from Dioscorea opposita or any extract naturally containing or containing after treatment one or more sapogenins (wild yam rhizome, agave leaf, which contains hecogenin and tigogenin, extracts of Liliacea plants and more particularly yucca or smilax containing smilagenin and sarsapogenin, or sarsaparilla) or Actigen Y from the company Actives Organics, or ginger.

Mention may also be made of DMAE (dimethyl MEA), extracts of sea fennel, of rockrose, of helichrysum, of aniseed, of paracress, and an extract of Acmella oleracea, for instance Gatuline® from Gattefossé.

### Anti-Glycation Agents

The term “anti-glycation agent” means a compound that prevents and/or reduces the glycation of skin proteins, in particular dermal proteins such as collagen.

Anti-glycation agents that may especially be mentioned include extracts of plants of the Ericacea family, such as an extract of blueberry (Vaccinium angustifolium or Vaccinium myrtillus), for example the product sold under the name Blueberry Herbasol Extract PG by the company Cosmetochem, ergothioneine and derivatives thereof, hydroxystilbenes and derivatives thereof, such as resveratrol and 3,3′,5,5′-tetrahydroxystilbene (these anti-glycation agents are described in patent applications FR 2 802 425, FR 2 810 548, FR 2 796 278 and FR 2 802 420, respectively), dihydroxystilbenes and derivatives thereof, polypeptides of arginine and of lysine such as the product sold under the name Amadorine® by the company Solabia, carsinine hydrochloride (sold by Exsymol under the name Alistin®), an extract of Helianthus annuus, for instance Antiglyskin® from Silab, wine extracts such as the extract of powdered white wine on a maltodextrin support sold under the name Vin blanc déshydraté 2F by the company Givaudan, thioctic acid (or alpha-lipoic acid), a mixture of extract of bearberry and of marine glycogen, for instance Aglycal LS 8777® from Laboratoires Sérobiologiques, and an extract of black tea, for instance Kombuchka® from Sederma, and mixtures thereof.

Agents for Stimulating the Synthesis of Dermal and/or Epidermal Macromolecules and/or for Preventing their Degradation

Among the active agents for stimulating the dermal macromolecules or for preventing their degradation, mention may be made of those acting:
either on collagen synthesis, such as extracts of Centella asiatica, asiaticosides and derivatives thereof; ascorbic acid or vitamin C and derivatives thereof; synthetic peptides such as iamin, biopeptide CL or palmitoyl oligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C®, sold by Exsymol; plant hormones such as auxins and lignans; folic acid; and an extract of Medicago sativa (alfalfa) such as the product sold by Silab under the name Vitanol®; a peptide extract of hazelnut such as the product sold by the company Solabia under the name Nuteline C®; and arginine;
or on the inhibition of collagen degradation, in particular agents acting on the inhibition of metalloproteases (MMP) more particularly such as MMP 1, 2, 3 and 9. Mention may be made of: retinoids and derivatives, extracts of Medicago sativa such as Vitanol® from Silab, an extract of Aphanizomenon flos-aquae (Cyanophyceae) sold under the name Lanablue® by Atrium Biotechnologies, oligopeptides and lipopeptides, lipoamino acids, the malt extract sold by the company Coletica under the trade name Collalift®; blueberry or rosemary extracts; lycopene; isoflavones, derivatives thereof or plant extracts containing them, in particular extracts of soybean (sold, for example, by the company Ichimaru Pharcos under the trade name Flavosterone SB®), of red clover, of flax or of kakkon; an extract of lychee sold by the company Cognis under the trade name Litchiderm LS 9704®; Dipalmitoyl Hydroxyproline sold by SEPPIC under the name Sepilift DPHP®: Baccharis genistelloides or Baccharine sold by Silab, an extract of moring a such as Arganyl LS 9781® from Cognis; the sage extract described in patent application FR-A-2 812 544 from the Labiatae family (Salvia officinalis from the company Flacksmann), an extract of rhododendron, a blueberry extract, and an extract of Vaccinium myrtillus such as those described in patent application FR-A-2 814 950;
or on the synthesis of molecules belonging to the elastin family (elastin and fibrillin), such as: retinol and derivatives, in particular retinyl palmitate; the extract of Saccharomyces cerevisiae sold by the company LSN under the trade name Cytovitin®; and the extract of the alga Macrocystis pyrifera sold by the company Secma under the trade name Kelpadelie®; a peptide extract of hazelnut such as the product sold by the company Solabia under the trade name Nuteline C®;
or on inhibition of elastin degradation, such as the peptide extract of seeds of Pisum sativum sold by the company LSN under the trade name Parelastyl®; heparinoids; and the N-acylamino amide compounds described in patent application WO 01/94381, such as {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethylphenylvalylglycine, or an ester thereof with a C1-C6 alcohol; an extract of rice peptides such as Colhibin® from Pentapharm, or an extract of Phyllanthus emblica such as Emblica® from Rona;
or on the synthesis of glycosaminoglycans, such as the product of fermentation of milk with Lactobacillus vulgaris, sold by the company Brooks under the trade name Biomin Yoghurt®) the extract of the brown alga Padina pavonica sold by the company Alban Müller under the trade name HSP30; the Saccharomyces cerevisiae extract available especially from the company Silab under the trade name Firmalift® or from the company LSN under the trade name Cytovitin®; an extract of Laminaria ochroleuca such as Laminaine® from Secma; essence of Mamaku from Lucas Meyer, and an extract of Cress (Odraline® from Silab);
or on the synthesis of fibronectin, such as the extract of the zooplankton Salina sold by the company Seporga under the trade name GP4G®; the yeast extract available especially from the company Alban Müller under the trade name Drieline®; and the palmitoyl pentapeptide sold by the company Sederma under the trade name Matrixyl®.

Among the active agents for stimulating epidermal macromolecules, such as fillagrin and keratins, mention may be made especially of the extract of Iupin sold by the company Silab under the trade name Structurine®; the extract of Fagus sylvatica beech buds sold by the company Gattefossé under the trade name Gatuline® RC; and the extract of the zooplankton Salina sold by the company Seporga under the trade name GP4G®; the copper tripeptide from Procyte; a peptide extract of Voandzeia substerranea such as the product sold by the company Laboratoires Sérobiologiques under the trade name Filladyn LS 9397®.

As preferred active agents for stimulating the synthesis of dermal and/or epidermal macromolecules and/or for preventing their degradation, mention may be made of synthetic peptides such as iamin, the biopeptide CL or palmitoyloligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C® sold by Exsymol; folic acid; an extract of Medicago sativa (alfalfa), such as the product sold by Silab under the name Vitanol®; a peptide extract of hazelnut, such as the product sold by the company Solabia under the name Nuteline C®; arginine; an extract of Aphanizomenon flos-aquae (Cyanophyceae) sold under the name Lanablue® by Atrium Biotechnologies, the malt extract sold by the company Coletica under the trade name Collalift®, lycopene; an extract of lychee; an extract of moring a such as Arganyl LS 9781® from Cognis; an extract of Vaccinium myrtillus such as those described in patent application FR-A-2 814 950; retinol and derivatives thereof, in particular retinyl palmitate; {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethylphenylvalylglycine, or an ester thereof with a C1-C6 alcohol; an extract of rice peptides such as Colhibin® from Pentapharm, or an extract of Phyllanthus emblica such as Emblica® from Rona; the extract of the brown alga Padina pavonica sold by the company Alban Müller under the trade name HSP30; the extract of Saccharomyces cerevisiae available especially from the company Silab under the trade name Firmalift® or from the company LSN under the trade name Cytovitin®; an extract of Laminaria ochroleuca such as Laminaine® from Secma; the essence of Mamaku from Lucas Meyer, the extract of lupin sold by the company Silab under the trade name Structurine®; the extract of Fagus sylvatica beech buds sold by the company Gattefosse under the trade name Gatuline® RC.

Agents for Stimulating Fibroblast or Keratinocyte Proliferation and/or Keratinocyte Differentiation

The agents for stimulating fibroblast proliferation that may be used in the composition according to the invention may be chosen, for example, from plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; and plant hormones such as gibberellins and cytokinins; a peptide extract of hazelnut such as the product sold by the company Solabia under the name Nuteline C®.

Preferably, an agent that promotes keratinocyte proliferation and/or differentiation will be used.

The agents for stimulating keratinocyte proliferation that may be used in the composition according to the invention especially comprise adenosine; phloroglucinol, the extract of Hydrangea macrophylla leaves, for instance Amacha Liquid E® from Ichimaru Pharcos, a yeast extract such as Stimoderm® from CLR; the extract of Larrea divaricata such as Capislow® from Sederma, mixtures of extract of papaya, of olive leaves and of lemon, such as Xyleine® from Vincience, the extract of Hydrangea macrophylla leaves, for instance Amacha Liquid E® from Ichimaru Pharcos, retinol and esters thereof, including retinyl palmitate, phloroglucinol, the nut cake extracts sold by the Gattefossé and the extracts of Solanum tuberosum such as Dermolectine® sold by Sederma.

Among the agents for stimulating keratinocyte differentiation are, for example, minerals such as calcium; sea fennel, a peptide extract of Iupin, such as the product sold by the company Silab under the trade name Structurine®; sodium beta-sitosteryl sulfate, such as the product sold by the company Seporga under the trade name Phytocohesine®; and a water-soluble extract of corn, such as the product sold by the company Solabia under the trade name Phytovityl®; a peptide extract of Voandzeia subterranea such as the product sold by the company Laboratoires Serobiologiques under the trade name Filladyn LS 9397®; and lignans such as secoisolariciresinol, and retinol and esters thereof, including retinyl palmitate.

As agents for stimulating keratinocyte proliferation and/or differentiation, mention may also be made of oestrogens such as oestradiol and homologues; cytokines.

As preferred active agents for stimulating fibroblast or keratinocyte proliferation and/or keratinocyte differentiation, mention will be made of plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; a peptide extract of hazelnut such as the product sold by the company Solabia under the name Nuteline C®; adenosine; phloroglucinol, a yeast extract such as Stimoderm® from CLR; a peptide extract of Iupin such as the product sold by the company Silab under the trade name Structurine®; a water-soluble corn extract, such as the product sold by the company Solabia under the trade name Phytovityl®; a peptide extract of Voandzeia subterranea, such as the product sold by the company Laboratoires Sérobiologiques under the trade name Filladyn LS 9397®; retinol and esters thereof, including retinyl palmitate.

### Agents for Promoting the Maturation of the Horny Envelope

Agents that participate in the maturation of the horny envelope, which becomes impaired with age and induces a decrease in transglutaminase activity, may be used in the compositions of the invention. Examples that may be mentioned include urea and derivatives thereof and in particular Hydrovance® from National Starch and the other active agents mentioned in L'Oréal patent application FR 2 877 220 (unpublished).

### NO-Synthase Inhibitors

The agent with an inhibitory action on NO synthase may be chosen from PCOs (procyannidol oligomers); plant extracts of the species Vitis vinifera sold especially by the company Euromed under the name “Leucocyanidines de raisins extra”, or by the company Indena under the name Leucoselect®, or finally by the company Hansen under the name “Extrait de marc de raisin”; plant extracts of the species Olea europaea preferably obtained from olive tree leaves and sold especially by the company Vinyals in the form of a dry extract, or by the company Biologia & Technologia under the trade name Eurol BT; and plant extracts of the species Gingko biloba, preferably a dry aqueous extract of this plant sold by the company Beaufour under the trade name “Ginkgo biloba extrait standard”, and mixtures thereof.

### Peripheral Benzodiazepine Receptor (PBR) Antagonists

Mention may be made, for example, of 1-(2-chlorophenyl)-N-(1-methylpropyl)-3-isoquinoline carboxamide; the compounds described in patent applications WO 03/030 937 and WO 03/068 753, pyridazino[4,5-b]indole-1-acetamide derivatives of general formula (VII) as described in document WO 00/44384.

### Agents for Increasing the Activity of the Sebaceous Glands

Mention may be made, for example, of methyl dehydrojasmonate, hecogenin, hedione and O-linoleyl-6D-glucose, and mixtures thereof.

### Agents for Stimulating the Energy Metabolism of Cells

The active agent for stimulating the energy metabolism of cells may be chosen, for example, from biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate, such as Sepitonic M3® from SEPPIC, and mixtures thereof; a beta-glucan derived from Saccharomyces cerevisiae, such as the product sold by the company Mibelle AG Biochemistry.

To complement and/or optimize the effects imparted by the cosmetic and/or dermatological active agents mentioned above on the keratin materials, it may be advantageous to incorporate into the compositions of the invention other additional ingredients, for instance matting agents, soft-focus fillers, and agents that promote the naturally pinkish coloration of the skin.

In particular, these additional ingredients may impart an immediate visual effect that will be relayed by the biological effect of the active agents mentioned above.

### Matting Agents

The term “matting agent” means agents intended to make the skin visibly more matt and less shiny.

The matting effect of the agent and/or composition containing it may especially be evaluated using a gonioreflectometer, by measuring the ratio R between the specular reflection and the scattered reflection. A value of R of less than or equal to 2 generally indicates a matting effect.

The matting agent may especially be chosen from a rice starch or a corn starch: INCI name: Zea mays (Corn) Starch, such as, in particular, the product sold under the trade name Farmal CS 3650 Plus 036500 by National Starch, kaolinite, talc, a pumpkin seed extract, cellulose microbeads, plant fibres, synthetic fibres, in particular polyamide fibres, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that may especially be mentioned include:
rice or corn starch, in particular an aluminium starch octenyl succinate sold under the name Dry Flo® by the company National Starch;
kaolinite;
silicas;
talc;
a pumpkin seed extract as sold under the name Curbilene® by the company Indena;
cellulose microbeads as described in patent application EP 1 562 562;
fibres, such as silk fibre, cotton fibre, wool fibre, flax fibre, cellulose fibre extracted especially from wood, from vegetables or from algae, polyamide fibre (Nylon®), modified cellulose fibre, poly-p-phenyleneterephthamide fibre, acrylic fibre, polyolefin fibre, glass fibre, silica fibre, aramid fibre, carbon fibre, Teflon® fibre, insoluble collagen fibre, polyester fibre, polyvinyl chloride or polyvinylidene chloride fibre, polyvinyl alcohol fibre, polyacrylonitrile fibre, chitosan fibre, polyurethane fibre, polyethylene phthalate fibre, fibres formed from a mixture of polymers, resorbable synthetic fibres, and mixtures thereof described in patent application EP 1 151 742;
expanded acrylic copolymer microspheres such as those sold by the company Expancel under the name Expancel 551®;
fillers with an optical effect as described in patent application FR 2 869 796, in particular:
polyamide powders (Nylon®), for instance Nylon 12 particles of the Orgasol type from Arkema, with a mean size of 10 microns and a refractive index of 1.54,
silica powders, for instance Silica beads SB150 from Miyoshi with a mean size of 5 microns and a refractive index of 1.45,
polytetrafluoroethylene powders, for instance PTFE Ceridust 9205F from Clariant, with a mean size of 8 microns and a refractive index of 1.36,
silicone resin powders, for instance the silicone resin Tospearl 145A from GE Silicone with a mean size of 4.5 microns and a refractive index of 1.41,
acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,
wax powders, for instance the paraffin wax particles Microease 114S from Micropowders, with a mean size of 7 microns and a refractive index of 1.54,
polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),
elastomeric crosslinked organopolysiloxane powders coated with silicone resin, especially with silsesquioxane resin, as described, for example, in patent U.S. Pat. No. 5,538,793. Such elastomer powders are sold under the names KSP-100, KSP-101, KSP-102, KSP-103, KSP-104 and KSP-105 by the company Shin-Etsu, and
talc/titanium dioxide/alumina/silica composite powders such as those sold under the name Coverleaf® AR-80 by the company Catalyst & Chemicals,
mixtures thereof,
compounds that absorb and/or adsorb sebum as described in patent application FR 2 869 796. Mention may be made especially of:
silica powders, for instance the porous silica microspheres sold under the name Silica Beads SB-700 sold by the company Miyoshi, the products Sunsphere® H51, Sunsphere® H33 and Sunsphere® H53 sold by the company Asahi Glass; the polydimethylsiloxane-coated amorphous silica microspheres sold under the name SA Sunsphere® H-33 and SA Sunsphere® H-53 sold by the company Asahi Glass;
amorphous mixed silicate powders, especially of aluminium and magnesium, for instance the product sold under the name Neusilin UFL2 by the company Sumitomo;
polyamide (Nylon®) powders, for instance Orgasol® 4000 sold by the company Arkema, and
acrylic polymer powders, especially of polymethyl methacrylate, for instance Covabead® LH85 sold by the company Wackherr; of polymethyl methacrylate/ethylene glycol dimethacrylate, for instance Dow Corning 5640 Microsponge® Skin Oil Adsorber sold by the company Dow Corning, or Ganzpearl®GMP-0820 sold by the company Ganz Chemical; of polyallyl methacrylate/ethylene glycol dimethacrylate, for instance Poly-Pore® L200 or Poly-Pore® E200 sold by the company Amcol; of ethylene glycol dimethacrylate/lauryl methacrylate copolymer, for instance Polytrap® 6603 sold by the company Dow Corning;
silicate particles, such as alumina silicate;
mixed silicate particles, such as:
magnesium aluminium silicate particles, such as saponite or hydrated magnesium aluminium silicate with a sodium sulfate sold under the trade name Sumecton® by the company Kunimine;
the magnesium silicate, hydroxyethylcellulose, black cumin oil, marrow oil and phospholipids complex or Matipure® from Lucas Meyer, and
mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Fillers with a Soft-Focus Effect

These fillers may be any material capable of modifying and hiding wrinkles by virtue of their intrinsic physical properties. These fillers may especially modify wrinkles via a tensioning effect, a covering effect or a soft-focus effect.

Examples of fillers that may be given include the following compounds:
porous silica microparticles, for instance the silica beads SB150 and SB700 from Miyoshi with a mean size of 5 μm; the series H Sunspheres® from Asahi Glass, for instance Sunspheres H33, H51 with respective sizes of 3.5 and 5 μm;
hollow hemispherical silicone resin particles such as NLK 500®, NLK 506® and NLK 510® from Takemoto Oil and Fat, especially described in EP-A-1 579 849;
silicone resin powders, for instance the silicone resin Tospearl® 145A from GE Silicone, with a mean size of 4.5 μm;
acrylic copolymer powders, especially of polymethyl (meth)acrylate, for instance the PMMA particles Jurymer MBI® from Nihon Junyoki, with a mean size of 8 μm, the hollow PMMA spheres sold under the name Covabead® LH85 by the company Wackherr, and vinylidene/acrylo-nitrile/methylene methacrylate expanded microspheres sold under the name Expancel®;
wax powders, for instance the paraffin wax particles MicroEase® 114S from MicroPowders, with a mean size of 7 μm;
polyethylene powders, especially comprising at least one ethylene/acrylic acid copolymer, for instance the Flobeads® EA 209 E particles from Sumitomo, with a mean size of 10 μm;
crosslinked elastomeric organopolysiloxane powders coated with silicone resin and especially with silsesquioxane resin, under the names KSP-100®, KSP1010, KSP-102®, KSP-103®, KSP-104® and KSP-105® by the company Shin-Etsu;
talc/titanium dioxide/alumina/silica composite powders, for instance those sold under the name Coverleaf AR-80® by the company Catalyst & Chemicals;
talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulfate, hydroxyapatite, calcium silicate, cerium dioxide and glass or ceramic microcapsules;
hydrophilic or hydrophobic, synthetic or unnatural, mineral or organic fillers such as silk fibres, cotton fibres, wool fibres, flax fibres, cellulose fibres extracted especially from wood, vegetables or algae, Polyamide (Nylon®) fibres, modified cellulose fibres, poly-p-terephthamide fibres, acrylic fibres, polyolefin fibres, glass fibres, silica fibres, aramid fibres, carbon fibres, polytetrafluoroethylene (Teflon®) fibres, insoluble collagen fibres, polyester fibres, polyvinyl chloride fibres, polyvinylidene chloride fibres, polyvinyl alcohol fibres, polyacrylonitriles fibres, chitosan fibres, polyurethane fibres, polyethylene phthalate fibres, fibres formed from a mixture of polymers, resorbable synthetic fibres, and mixtures thereof described in patent application EP 1 151 742;
spherical elastomeric crosslinked silicones, for instance Trefil E-505C® or E-506C® from Dow Corning;
abrasive fillers, which, via a mechanical effect, smooth out the skin microrelief, such as abrasive silica, for instance Abrasif SP® from Semanez or nutshell powders (for example of apricot or walnut, from Cosmetochem).

The fillers with an effect on the signs of ageing are especially chosen from porous silica microparticles, hollow hemispherical silicones, silicone resin powders, acrylic copolymer powders, polyethylene powders, crosslinked elastomeric organopolysiloxane powders coated with silicone resin, talc/titanium dioxide/alumina/silica composite powders, precipitated calcium carbonate, magnesium carbonate, magnesium hydrogen carbonate, barium sulfate, hydroxyapatite, calcium silicate, cerium dioxide, glass or ceramic microcapsules, and silk fibres or cotton fibres, and mixtures thereof.

The filler may be a soft-focus filler.

The term “soft-focus” filler means a filler which in addition gives the complexion transparency and a hazy effect. Preferably, the soft-focus fillers have a mean particle size of less than or equal to 15 microns. These particles may be in any form and in particular may be spherical or non-spherical. These fillers are more preferably non-spherical.

The soft-focus fillers may be chosen from silica and silicate powders, especially alumina powder, powders of polymethyl methacrylate (PMMA) type, talc, silica/TiO2 or silica/zinc oxide composites, polyethylene powders, starch powders, polyamide powders, styrene/acrylic copolymer powders and silicone elastomers, and mixtures thereof.

Mention may be made in particular of talc with a number-average size of less than or equal to 3 microns, for example talc with a number-average size of 1.8 microns and especially the product sold under the trade name Talc P3® by the company Nippon Talc, Nylon® 12 powder, especially the product sold under the name Orgasol 2002 Extra D Nat Cos® by the company Atochem, silica particles 1% to 2% surface-treated with a mineral wax (INCI name: hydrated silica (and) paraffin) such as the products sold by the company Degussa, amorphous silica microspheres, such as the products sold under the name Sunsphere, for example of reference H-53® by the company Asahi Glass, and silica microbeads such as those sold under the name SB-700® or SB-150® by the company Miyoshi, this list not being limiting.

The concentration of these fillers with an effect on the signs of ageing in the compositions according to the invention may be between 0.1% and 40%, or even between 0.1% and 20% by weight, relative to the total weight of the composition.

### Agents for Promoting the Naturally Pinkish Coloration of the Skin

The compositions used according to the invention may also comprise an agent for promoting the naturally pinkish coloration of the skin. Mention may be made especially of:
a self-tanning agent, i.e. an agent which, when applied to the skin, especially to the face, can produce a tan effect that is more or less similar in appearance to that which may result from prolonged exposure to the sun (natural tan) or under a UV lamp;
an additional colouring agent, i.e. any compound that has particular affinity for the skin, which allows it to give the skin a lasting, non-covering coloration (i.e. that does not have a tendency to opacify the skin) and that is not removed either with water or using a solvent, and that withstands both rubbing and washing with a solution containing surfactants. Such a lasting coloration is thus distinguished from the superficial and transient coloration provided, for example, by a makeup pigment; and mixtures thereof.

Examples of self-tanning agents that may especially be mentioned include:

dihydroxyacetone (DHA) (used as a supplement to a photoprotective (or screening) or skincare compound C),
erythrulose, and
the combination of a catalytic system formed from:
manganese and/or zinc oxide salts, and
alkali metal and/or alkaline-earth metal hydrogen carbonates.

The self-tanning agents are generally chosen from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in patent application FR 2 466 492 and WO 97/35842, dihydroxyacetone (DHA) and 4,4-dihydroxypyrazolin-5-one derivatives as described in patent application EP 903 342. DHA will preferably be used.

DHA may be used in free and/or encapsulated form, for example in lipid vesicles such as liposomes, especially described in patent application WO 97/25970.

In general, the self-tanning agent is present in an amount ranging from 0.01% to 20% by weight and preferably in an amount of between 0.1% and 10% of the total weight of the composition.

Other dyes that allow modification of the colour produced by the self-tanning agent may also be used.

These dyes may be chosen from synthetic or natural direct dyes.

These dyes may be chosen, for example, from red or orange dyes of the fluorane type such as those described in patent application FR 2 840 806. Mention may be made, for example, of the following dyes:
tetrabromofluoresceine or eosin known under the CTFA name: CI-45380 or Red 21
phloxin B known under the CTFA name: CI-45410 or Red 27
diiodofluoresceine known under the CTFA name: CI-45425 or Orange 10;
dibromofluoresceine known under the CTFA name: CI-45370 or Orange 5;
the sodium salt of tetrabromofluoresceine known under the CTFA name: CI-45380 (Na salt) or Red 22;
the sodium salt of phloxin B known under the CTFA name: CI-45410 (Na salt) or Red 28;
the sodium salt of diiodofluoresceine known under the CTFA name: CI-45425 (Na salt) or Orange 11;
erythrosine known under the CTFA name: CI-45430 or Acid Red 51.
phloxin known under the CTFA name: CI-45405 or Acid Red 98.

These dyes may also be chosen from anthraquinones, caramel, carmine, carbon black, azulene blues, methoxalene, trioxalene, guajazulene, chamuzulene, rose Bengal, eosin 10B, cyanosin and daphinin.

These dyes may also be chosen from indole derivatives, for instance the monohydroxyindoles as described in patent FR 2 651 126 (i.e.: 4-, 5-, 6- or 7-hydroxyindole) or the dihydroxyindoles as described in patent EP-B-0 425 324 (i.e.: 5,6-dihydroxyindole, 2-methyl-5,6-dihydroxyindole, 3-methyl-5,6-dihydroxyindole or 2,3-dimethyl-5,6-dihydroxyindole).

The examples below are given as non-limiting illustrations of the field of the invention.

### EXAMPLES

### 1) Examples of Preparation of a Film According to the Invention

Formula A below is Prepared:

| | |
|---|---|
| Pebax 2533 SA 01 (Arkema) | 5% |
| Ethanol | 95% |

The formula is left at 60° C. for 15 days to ensure good dissolution. After returning to room temperature, it may be applied to the skin or the hair.

The spreading is very easy and the solution dries. No tautness of the skin is felt, and the coating formed is imperceptible to the eye.

After 4 hours, a check may be made to see that the coating is still intact or virtually intact. To do this, one of the edges is lifted up. It is seen that the coating, which is extremely thin (less than 5 μm), is virtually intact.

In another test, the following examination is performed:

Bath for 20 minutes, followed by drying. 4 hours later, the same manipulation for checking the integrity of the coating is performed.

The coating is virtually intact.

The same tests were performed with film-forming materials such as Resin 28-29-30 (Akzo-Nobel) or elastomeric resins such as Baycusan C1004 (Bayer).

In the first case, the coating does not withstand either of the two tests.

In the second case, the coating according to the invention withstands relatively well the first of the two tests, but does not withstand the second test. Strong detachment is in particular noted.

Since the coating is slightly non-slip, this aspect can be corrected, if so desired.

In this case, the polymer may be combined with a third compound, for instance a polyamide resin with amine end groups, for instance the product sold under the trade name Versamid 756 (Cognis).

### Formula B Below is Prepared:

| | |
|---|---|
| Pebax 2533 SA 01 (Arkema) | 5% |
| Polyamide resin with amine end groups | 10% |
| (VERSAMID 756 from Cognis) | |
| Ethanol | 85% |

If it is desired to give the film a little sheen, without losing its qualities, the polymer may be combined with a resin, especially an acrylic resin.

### Formula C Below is Prepared:

| | |
|---|---|
| Pebax 2533 SA 01 (Arkema) | 5% |
| Resin 28-29-30 (Akzo-Nobel) - Vinyl acetate/crotonic | 5% |
| acid/vinyl neodecanoate terpolymer | |
| Ethanol | 90% |

### 2) Antiperspirant Skin Treatment Formulations

Formulation A may be used on the skin in the form obtained.

Formulation B is particularly advantageous since it does not have any non-slip effect, it better withstands the inevitable friction in the region of the armpits.

Agents such as powders, talc, perlite, antimicrobial active agents or antiperspirant active agents such as aluminium or zirconium salts may also be introduced into the formulation.

It is also possible to apply, in a first stage, formulation A, and then an aqueous formulation of an aluminium salt. In the odour tests, it is noted that this process functions just as well as direct application of the aluminium salt to the skin.

It is also possible to apply an antiperspirant composition of an aluminium salt in a first stage, and then to apply formulation A or B.

### 3) Hair Dyeing Formulation

Formulation C is applied to the edges of the hair. A lightening oxidation dye Majirel 3.1 is then applied to the hair.

Staining of the scalp is thus avoided.

The use of a formulation D, characterized in that it does not contain the polymer according to the invention, does not give good stain-protection results.

| | |
|---|---|
| Resin 28-29-30 (Akzo-Nobel) - Vinyl acetate/crotonic | 10% |
| acid/vinyl neodecanoate terpolymer | |
| Ethanol | 90% |

### 4) Lip Makeup Formulation

A colouring formulation comprising a red anionic dye (disodium salt of Ponceau SX-Inci UE 14700 from the company LCW (Sensient) 0.5%, water (80%) and ethanol (9.5%) is applied.

After drying, and while avoiding wetting the lips, formulation A is applied to the left part of the lips.

After one hour, it is seen that the colour is well protected since the lips remain well coloured on the left side, whereas the colour disappears in less than one hour on the right side.

### 5) Skin Photoprotectinq Formulation

The Following Antisun Formulation is Prepared:

| | |
|---|---|
| Pebax 2533 SA 01 (Arkema) | 5% |
| Terephthalylidenedicamphorsulfonic acid, | 3% |
| (Mexoryl SX) | |
| Ethanol | 92% |

This formulation is specially suited to daily photoprotection since it withstands movements without being uncomfortable. Furthermore, it is water-resistant and thus allows the use of water-soluble screening agents.

### 6) Hair Formulation

Formulation A, B or C as defined is applied to the hair.

To do this, the dry hair is rubbed with one of these formulations, in a proportion of about 6 g per head hair.

Formulation A gives the best results as regards the styling effect without the hairs sticking together.

Formulation B gives a styling effect with a small amount of fixing (sticking-together of the hairs).

Formulation C gives a styling effect with small amount of fixing and allows easy removal on washing. Thus, it is suitable for use as a spray (pressurized or in a pump-dispenser bottle) and for obtaining fixing effects. According to the system of pulverization used, the total concentration must be adjusted. Typically, ethanol will be diluted with a factor 2.

### 7) Skin Fragrancing Formulation

Trésor from Lancôme is applied to the skin, followed by application of formulation A as defined previously.

A formulation similar to formulation A also containing an additive for fragmenting or detaching the formed film may also be applied. Thus, by means of this process, it is possible to benefit from slow diffusion, and the release of the fragrance can be activated by scratching or detaching the coating.

1. Cosmetic process for treating a keratin material, characterized in that it comprises at least one step of applying to the said keratin material a coating formed from a liquid composition comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent.

2. Process according to claim 1, in which the polyamide-polyether polymer is capable of leading, by drying at room temperature and at a relative humidity of 55%, to a material with a mechanical profile defined by at least:
a degree of elongation at break (ε) of greater than or equal to 150%,
an instantaneous recovery (Rᵢ) of greater than or equal to 75% after an elongation of 150%,
a recovery at 300 seconds (R₃₀₀ₛ) of greater than 80%, after an elongation of 150%.

3. Process according to claim 1, in which the copolymers containing polyamide blocks and polyether blocks are chosen from those comprising the following blocks:
1) polyamide blocks bearing diamine chain ends with polyoxyalkylene blocks bearing dicarboxylic chain ends.
2) polyamide blocks bearing dicarboxylic chain ends with polyoxyalkylene blocks bearing diamine chain ends obtained by cyanoethylation and hydrogenation of α,ω-dihydroxylated aliphatic polyoxyalkylene blocks known as polyether diols.
3) polyamide blocks containing dicarboxylic chain ends with polyether diols.

4. Process according to claim 3, in which the copolymer containing polyamide blocks and polyether blocks is chosen from copolymers containing polyamide-6 or polyamide-12 blocks and polyethylene glycol or polytetramethylene glycol blocks.

5. Process according to claim 1, in which the solvent(s) are chosen from linear C₁-C₄ monoalcohols; mixtures of water and of a linear C₁-C₄, monoalcohol, acetone, liquefied gases such as dimethyl ether, C₁-C₃₀ alkanes; liquid C₈-C₁₄ fatty alcohols; amide oils; C₃-C₈ alkylene carbonates.

6. Process according to claim 1, in which the composition comprising the copolymer(s) containing polyamide blocks and polyether blocks also comprises at least one agent for aiding or modifying the properties of the coating.

7. Process according to claim 1, comprising
(i) an additional step for drying the copolymer(s) containing polyamide blocks and polyether blocks, such as the application of a supply of energy, or
(ii) an additional step for facilitating the removal of the coating, or
(iii) a step of pretreatment of the keratin materials and/or a step of pretreatment of the composition containing the copolymer containing polyamide blocks and polyether blocks.

8. Cosmetic assembly comprising:
a) at least one liquid composition as claimed in claim 1,
b) a device for applying the said composition to the keratin material to be treated.

9. Process according to claim 1, which comprises:
a) applying to the said keratin material a cosmetic composition (A) comprising at least one agent for treating a keratin material;
b) optionally rinsing and/or drying;
c) applying to the said human keratin material a coating formed in situ from a liquid composition (B) comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent.

10. Process according to claim 1 which comprises:
a) applying to the said human keratin material a coating formed in situ from a liquid composition (B) comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent;
b) optionally rinsing and/or drying;
c) applying to the said human keratin material a cosmetic composition (A) comprising at least one agent for treating keratin materials.

11. Process according to claim 9, wherein the agent(s) for treating keratin materials are chosen from haircare agents such as antidandruff agents and hair conditioners; agents for participating in hair dyeing, such as oxidation dye precursors, direct dyes, reducing agents and oxidizing agents; haircare active agents; agents for colouring the skin, the lips, the face, the nails, the eyelashes or the eyebrows, such as self-tanning agents, pigments, nacres or direct dyes; sheen agents; matting agents; agents for producing optical effects; fluorescers; organic UV-screening agents, mineral UV-screening agents, perspiration-treatment agents, deodorant active agents and fragrances.

12. Cosmetic assembly comprising at least:
a) a first composition (A) comprising at least one agent for treating a keratin materials;
b) a second liquid composition (B) comprising, in a cosmetically acceptable medium, at least one copolymer containing polyamide blocks and polyether blocks resulting from the co-polycondensation of polyamide blocks containing reactive ends with polyether blocks containing reactive ends; the said copolymer being in solution or in dispersion, and at least one organic solvent.

13. Process according to claim 2, in which the copolymers containing polyamide blocks and polyether blocks are chosen from those comprising the following blocks:
1) polyamide blocks bearing diamine chain ends with polyoxyalkylene blocks bearing dicarboxylic chain ends.
2) polyamide blocks bearing dicarboxylic chain ends with polyoxyalkylene blocks bearing diamine chain ends obtained by cyanoethylation and hydrogenation of α,ω-dihydroxylated aliphatic polyoxyalkylene blocks known as polyether diols.
3) polyamide blocks containing dicarboxylic chain ends with polyether diols.

14. Process according to claim 2, in which the solvent(s) are chosen from linear C₁-C₄ monoalcohols; mixtures of water and of a linear C₁-C₄, monoalcohol, acetone, liquefied gases such as dimethyl ether, C₁-C₃₀ alkanes; liquid C₈-C₁₄ fatty alcohols; amide oils; C₃-C₈ alkylene carbonates.

15. Process according to claim 3, in which the solvent(s) are chosen from linear C₁-C₄ monoalcohols; mixtures of water and of a linear C₁-C₄, monoalcohol, acetone, liquefied gases such as dimethyl ether, C₁-C₃₀ alkanes; liquid C₈-C₁₄ fatty alcohols; amide oils; C₃-C₈ alkylene carbonates.

16. Process according to claim 4, in which the solvent(s) are chosen from linear C₁-C₄ monoalcohols; mixtures of water and of a linear C₁-C₄, monoalcohol, acetone, liquefied gases such as dimethyl ether, C₁-C₃₀ alkanes; liquid C₈-C₁₄ fatty alcohols; amide oils; C₃-C₈ alkylene carbonates.

17. Process according to claim 2, in which the composition comprising the copolymer(s) containing polyamide blocks and polyether blocks also comprises at least one agent for aiding or modifying the properties of the coating.

18. Process according to claim 3, in which the composition comprising the copolymer(s) containing polyamide blocks and polyether blocks also comprises at least one agent for aiding or modifying the properties of the coating.

19. Process according to claim 4, in which the composition comprising the copolymer(s) containing polyamide blocks and polyether blocks also comprises at least one agent for aiding or modifying the properties of the coating.

20. Process according to claim 5, in which the composition comprising the copolymer(s) containing polyamide blocks and polyether blocks also comprises at least one agent for aiding or modifying the properties of the coating.
